(19) United States
(12) Patent Application Publication
(10) Pub. No.: US 2026/0265308 A1
(43) Pub. Date: Sep. 10, 2026

(54) AAV9 CAPSID VARIANTS ESCAPING NEUTRALIZING HUMAN ANTIBODIES

(71) Applicant: University of Florida Research Foundation, Incorporated, Gainesville, FL (US)

(72) Inventors: Robert McKenna, Gainesville, FL (US); Mario Mietzsch, Gainesville, FL (US); Jane Hsi, Gainesville, FL (US); Austin Nelson, Gainesville, FL (US)

(73) Assignee: University of Florida Research Foundation, Incorporated, Gainesville, FL (US)

(21) Appl. No.: 19/154,826

(22) PCT Filed: Feb. 8, 2024
(86) PCT No.: PCT/US2024/015006

§ 371 Date: Aug. 8, 2025

Related U.S. Application Data
(60) Provisional application No. 63/444,325, filed on Feb. 9, 2023.

(51) Int. Cl. C07K 14/005; C07K 16/081; C12N 15/86

(52) U.S. Cl. CPC C07K 14/005; C07K 16/081; C12N 15/86; C07K 2317/21; C07K 2317/33; C07K 2317/55; C07K 2317/76; C12N 2750/14122; C12N 2750/14143

(57) ABSTRACT

This disclosure relates to variant AAV9 particles engineered to escape host neutralizing antibodies but retain or improve transduction efficiency, production efficiency and/or infectivity, and their use as gene delivery vehicles.

### CROSS-REFERENCE TO RELATED APPLICATIONS

This application claims the benefit under 35 U.S.C. § 119(e) of U.S. Provisional Application No. 63/444,325, filed on Feb. 9, 2023, entitled “AAV9 CAPSID VARIANTS ESCAPING NEUTRALIZING HUMAN ANTIBODIES,” the entire disclosure of which is incorporated by reference herein.

### GOVERNMENT SUPPORT

This invention was made with government support under Grant Number R01 GM082946, awarded by the National Institutes of Health. The government has certain rights in the invention.

### REFERENCE TO AN ELECTRONIC SEQUENCE LISTING

The contents of the electronic sequence listing (U119670105WO00-SEQ-AXW.xml; Size: 10,977 bytes; and Date of Creation: Feb. 6, 2024) is herein incorporated by reference in its entirety.

### BACKGROUND

The use of vectors based on adeno-associated virus serotype 9 (AAV9) for gene therapy applications has grown rapidly over recent years, especially following the FDA approval of Zolgensma®, an AAV9-based biologic for the treatment of spinal muscular atrophy. In addition, there is interest in broader AAV9 usage due to (1) its ability to transduce cardiac and skeletal muscle, liver, pancreas, the eye, and (2) its capability to cross the blood-brain-barrier to transduce the central nervous system. However, the presence of pre-existing neutralizing antibodies against AAV9 capsids in a large percentage of the population excludes many patients from being treated. Further, cross-reactivity commonly occurs between variants, such that a neutralizing antibody against a first AAV serotype vector may also neutralize a second AAV serotype vector. When host neutralizing antibodies are encountered, treatment of a disease using AAV particles which are neutralized by the host's neutralizing antibodies can cause detrimental immunogenic effects.

### SUMMARY

The inventors of this disclosure have discovered that certain AAV serotypes (e.g., AAV9) cross-react with pre-existing neutralizing antibodies in some subjects. Up to 40% of the population has neutralizing antibodies against AAV9. For some AAV serotypes, it is even higher. For example, about 60% of the population has neutralizing antibodies against AAV2.

In some embodiments, anti-capsid antibodies against other AAV serotypes (e.g., AAV8, AAVrh.10) cross-react with and neutralize AAV9. This is likely due in part to the structural similarity in some surface loops between the neutralizing antibodies (e.g., AAV8, AAVrh.10) and the AAV serotype of choice for a specific treatment (e.g., AAV9). In addition to neutralizing antibodies against other AAV serotypes which cross-react with AAV9, use of AAV9-based biologics, such as an AAV-based biologic used to treat spinal muscular atrophy, can also lead patients to develop neutralizing antibodies against AAV9. This limits the availability of AAV9-based therapeutics as a treatment option for those patients, as such treatment can cause detrimental immunogenic effects. Patients who test positive against the AAV serotype of choice, or even a cross-reactive AAV serotype, for a specific treatment have to be excluded from the cohort.

In order to circumvent the problems of antibody neutralization of certain AAV serotypes with therapeutic promise (e.g., AAV9), the inventors of this disclosure have engineered variant recombinant AAV9 particles that have one or more mutations at one or more amino acid positions in one or more capsid proteins that enable the variant particles to escape neutralizing antibodies, while retaining (e.g., at least partially), for example without diminishing, or, in some embodiments, improving the transduction efficiency, production efficiency, and/or infectivity.

Recombinant AAV9 particles disclosed herein can be used in therapeutic applications for patients that test positive for neutralizing antibodies against different AAV serotypes (e.g., AAV8, AAVrh.10). These patients would otherwise be excluded from a therapeutic regimen comprising AAV9 particle-delivered gene therapy (e.g., due to cross-reactivity). The disclosed AAV9 gene therapy particles that escape from pre-existing antibodies against other serotypes also provide an alternative serotype for the treatment of patients previously treated with an AAV of another serotype (e.g., AAV8, AAVrh.10). These AAV9 particles can also be used for treating patients previously treated with an AAV vector of the same serotype (e.g., an AAV9 vector),

Accordingly, in some embodiments a composition as described herein is administered to a patient having, suspected of having, or at risk of developing neutralizing antibodies against AAV9 and/or an AAV serotype that cross-reacts with AAV9 (e.g., AAV8, AAVrh.10). In some embodiments, the patient was previously administered an AAV9-based therapeutic (e.g., a recombinant AAV9 particle comprising a therapeutic cargo, e.g., a nucleic acid sequence encoding a survival motor neuron protein), and is re-dosed with a recombinant AAV9 particle of the disclosure (e.g., a recombinant AAV9 particle which differs from that of the first dosing, but still comprises the same therapeutic cargo, e.g., a nucleic acid sequence encoding a survival motor neuron protein). In some embodiments, the patient was previously administered an AAV-based therapeutic of a serotype which cross-reacts with AAV9 (e.g., a recombinant AAV8 or AAVrh.10 particle comprising a therapeutic cargo, e.g., a nucleic acid sequence encoding a survival motor neuron protein), and is re-dosed with a recombinant AAV9 particle of the disclosure (e.g., a recombinant AAV9 particle comprising the same therapeutic cargo as the first dose, e.g., a nucleic acid sequence encoding a survival motor neuron protein).

Use of the disclosed variant AAV9 particles that escape pre-existing antibodies in clinical trials has the potential to increase the patient cohort that can be enrolled in clinical trials, thus enabling gene therapy in a much larger percentage of the population.

In some aspects, the present disclosure provides variant capsid proteins and particles having one or more mutations (e.g., amino acid substitutions, deletions, and/or insertions) relative to a wildtype AAV9 capsid particle.

Aspects of the disclosure relate to a recombinant adeno-associated virus 9 (rAAV9) particle comprising a capsid protein comprising one or more mutations, wherein said mutations result in modulated reactivity to a neutralizing antibody, altered or unchanged production efficiency, and/or altered or unchanged infectivity, relative to a wild-type AAV9 particle having a capsid protein with an amino acid sequence of SEQ ID NO: 2.

In some embodiments, the neutralizing antibody comprises one or more Fragment antigen-binding (Fab) region(s) which binds AAV9. In some embodiments, the neutralizing antibody is against AAV9. In some embodiments, the neutralizing antibody is against AAV1, AAV2, AAV2.5, AAV3, AAV4, AAV5, AAV6, AAV7, AAV8, AAVrh.10, AAVrh.74, AAV10, AAV11, AAV12, AAV-DJ, PHP.eB, AvianAAV, or AAV13.

In some embodiments, the neutralizing antibody is Immunoglobulin G (IgG), Immunoglobulin M (IgM), Immunoglobulin A (IgA), Immunoglobulin D (IgD), or Immunoglobulin E (IgE). In some embodiments, the neutralizing antibody is IgG. In some embodiments, the neutralizing antibody is a preexisting neutralizing antibody against AAV9. In some embodiments, the neutralizing antibody is generated in a subject in response to administration of an AAV vector. In some embodiments, the AAV vector is an AAV9 vector. In some embodiments, the neutralizing antibody is isolated from the subject. In some embodiments, the neutralizing antibody comprises one or more Fragment antigen-binding (Fab) region(s) which binds AAV9.

In some embodiments, the AAV vector comprises a nucleic acid sequence encoding a therapeutic molecule.

In some embodiments, the reactivity to neutralizing antibodies is decreased, relative to wild-type AAV9 particles. In some embodiments, the reactivity to neutralizing antibodies is decreased by 20-100%, relative to wild-type AAV9 particles. In some embodiments, the reactivity to neutralizing antibodies is decreased by 75-100%, relative to wild-type AAV9 particles. In some embodiments, the production efficiency is unchanged, relative to wild-type AAV9 particles. In some embodiments, the production efficiency is increased, relative to wild-type AAV9 particles. In some embodiments, the production efficiency is increased by 50-400%, relative to wild-type AAV9 particles. In some embodiments, the infectivity is unchanged, relative to wild-type AAV9 particles. In some embodiments, the infectivity is increased, relative to wild-type AAV9 particles. In some embodiments, the infectivity is increased by 50-400%, relative to wild-type AAV9 particles. In some embodiments, the rAAV9 particle retains at least 50% infectivity, relative to wild-type AAV9 particles, relative to wild-type AAV9 particles.

In some embodiments, the capsid protein is one or more of the capsid proteins selected from the group consisting of: VP1, VP2 and VP3.

In some embodiments, a capsid protein comprises one or more mutations that can be amino acid substitutions, deletions, and/or insertions (e.g., relative to a wild-type AAV9 capsid protein sequence, for example relative to SEQ ID NO: 2). In some embodiments, the one or more mutations are at amino acid positions selected from the group consisting of: D327, N329, D441, T491, V493, R533, N552, V553, D556, 1560, N562, Q588, W595, Q597, A656, P658, Y706, and N709. In some embodiments, the one or more mutations are amino acid substitution(s) selected from the group consisting of: D327R, D327T, N329D, D441N, T491R, V493P, R533S, N552A, V553A, D556P, I560F, N562Y, Q588R, W595G, Q597S, A656G, P658L, Y706D, N709I, and N709Y.

In some embodiments, an amino acid substitution is D327R. In some embodiments, an amino acid substitution is D441N. In some embodiments, an amino acid substitution is T491R. In some embodiments, an amino acid substitution is V493P. In some embodiments, an amino acid substitution is R533S. In some embodiments, an amino acid substitution is N552A. In some embodiments, an amino acid substitution is V553A. In some embodiments, an amino acid substitution is D556P. In some embodiments, an amino acid substitution is I560F. In some embodiments, an amino acid substitution is N562Y. In some embodiments, an amino acid substitution is Q588R. In some embodiments, an amino acid substitution is Y706D. In some embodiments, an amino acid substitution is N709I. In some embodiments, an amino acid substitution is N709Y. In some embodiments, the amino acid substitutions are D327T and N329D. In some embodiments, the amino acid substitutions are N552A and V553A. In some embodiments, the amino acid substitutions are W595G and Q597S. In some embodiments, the amino acid substitutions are A656G and P658L. In some embodiments, the amino acid substitutions are Y706D and N709I. In some embodiments, the amino acid substitutions are Q588R and T491R.

In some embodiments, the rAAV9 particle further comprises a transgene comprising a gene of interest. In some embodiments, the gene of interest comprises a survival motor neuron (SMN) gene. In some embodiments, the SMN gene is a SMN1 or SMN2 gene.

In some embodiments, the gene of interest encodes a therapeutic protein. In some embodiments, the therapeutic protein is an antibody, a peptibody, a growth factor, a clotting factor, a hormone, a membrane protein, a cytokine, a chemokine, an activating or inhibitory peptide acting on cell surface receptors or ion channels, a cell-permeant peptide targeting intracellular processes, a thrombolytic, an enzyme, a bone morphogenetic protein, a nuclease or other protein used for gene editing, an Fc-fusion protein, or an anticoagulant. In some embodiments, the therapeutic protein is a survival motor neuron (SMN) protein.

In some embodiments, the gene of interest encodes a detectable molecule. In some embodiments, the detectable molecule is a fluorescent protein, a bioluminescent protein, or a protein that provides color, or a fragment thereof.

Aspects of the disclosure relate to a composition comprising a rAAV9 particle of any one of the preceding embodiments. In some embodiments, the composition further comprises a pharmaceutically acceptable carrier.

Aspects of the disclosure relate to a method of delivering a protein of interest to a subject. In some aspects, the method comprises administering to the subject a composition comprising a rAAV9 particle of any one of the previous aspects and/or embodiments, wherein the gene of interest encodes a protein of interest. In some embodiments, the protein of interest is a therapeutic protein or detectable molecule. In some embodiments, the therapeutic protein is an antibody, a peptibody, a growth factor, a clotting factor, a hormone, a membrane protein, a cytokine, a chemokine, an activating or inhibitory peptide acting on cell surface receptors or ion channels, a cell-permeant peptide targeting intracellular processes, a thrombolytic, an enzyme, a bone morphogenetic protein, a nuclease or other protein used for gene editing, an Fc-fusion protein, or an anticoagulant. In some embodiments, the therapeutic protein is a survival motor neuron (SMN) protein. In some embodiments, the detectable molecule is a fluorescent protein, a bioluminescent protein, or a protein that provides color, or a fragment thereof.

In some embodiments, the subject is a mammalian subject. In some embodiments, the mammalian subject is a human, a non-human primate, non-primate mammal, or mouse. In some embodiments, the subject has or is suspected of having a disease or disorder selected from the group consisting of: cancer, diabetes, autoimmune disease, kidney disease, cardiovascular disease, pancreatic disease, intestinal disease, liver disease, hemophilia, α1-antitrypsin (AAT) deficiency, ischemia, skeletal disease, muscular disease and pulmonary disease. In some embodiments, the subject has or is suspected of having spinal muscular atrophy (SMA).

In some embodiments, administration of the rAAV9 particle to the subject results in the prevention, alleviation or amelioration of one or more signs or symptoms of the disease or disorder. In some embodiments, the rAAV9 particle is administered to the subject subcutaneously, intraocularly, intravitreally, subretinally, parenterally, intravenously (IV), intracerebro-ventricularly, intramuscularly, intrathecally (IT), intracisternally, orally, intraperitoneally, by oral or nasal inhalation, or by direct injection to one or more cells, tissues, or organs of the subject.

Aspects of the disclosure relate to a vector comprising one or more nucleic acid encoding Cap proteins, wherein the Cap proteins form a rAAV9 particle of any one of the previous aspects and/or embodiments when the vector is used to form capsids. In some embodiments, the capsid protein comprises one or more mutations (e.g., amino acid substitutions, deletions, and/or insertion) relative to SEQ ID NO: 2.

Aspects of the disclosure relate to a kit comprising a vector comprising one or more nucleic acid encoding Cap proteins, wherein the Cap proteins form a rAAV9 particle of any one of the previous aspects and/or embodiments when the vector is used to form capsids, and a vector comprising AAV helper genes, wherein the vector comprising one or more nucleic acid encoding Cap proteins and the vector comprising AAV helper genes are provided in a first and second container, wherein the first and second containers are different. In some embodiments, the kit further comprises AAV packaging cells that are provided in a third container that is separate from the first and second containers. In some embodiments, the AAV helper genes encode E1, E2, E4 and/or VA helper proteins.

Aspects of the disclosure relate to a capsid protein of serotype AAV9 comprising one or more mutations. In some embodiments, a mutation comprises an amino acid substitution, deletion, or insertion. In some embodiments, the capsid protein of serotype AAV9 comprises a mutation in the D327 amino acid position in SEQ ID NO: 2. In some embodiments, the mutation is D327R.

In some embodiments, the capsid protein of serotype AAV9 comprises a mutation in the D441 amino acid position in SEQ ID NO: 2. In some embodiments, the mutation is D441N.

In some embodiments, the capsid protein of serotype AAV9 comprises a mutation in the T491 amino acid position in SEQ ID NO: 2. In some embodiments, the mutation is T491R.

In some embodiments, the capsid protein of serotype AAV9 comprises a mutation in the V493 amino acid position in SEQ ID NO: 2. In some embodiments, the mutation is V493P.

In some embodiments, the capsid protein of serotype AAV9 comprises a mutation in the R533 amino acid position in SEQ ID NO: 2. In some embodiments, the mutation is R533S.

In some embodiments, the capsid protein of serotype AAV9 comprises a mutation in the N552 amino acid position in SEQ ID NO: 2. In some embodiments, the mutation is N552A.

In some embodiments, the capsid protein of serotype AAV9 comprises a mutation in the V553 amino acid position in SEQ ID NO: 2. In some embodiments, the mutation is V553A.

In some embodiments, the capsid protein of serotype AAV9 comprises a mutation in the D556 amino acid position in SEQ ID NO: 2. In some embodiments, the mutation is D556P.

In some embodiments, the capsid protein of serotype AAV9 comprises a mutation in the I560 amino acid position in SEQ ID NO: 2. In some embodiments, the mutation is I560F.

In some embodiments, the capsid protein of serotype AAV9 comprises a mutation in the N562 amino acid position in SEQ ID NO: 2. In some embodiments, the mutation is N562Y.

In some embodiments, the capsid protein of serotype AAV9 comprises a mutation in the Q588 amino acid position in SEQ ID NO: 2. In some embodiments, the mutation is Q588R.

In some embodiments, the capsid protein of serotype AAV9 comprises a mutation in the Y706 amino acid position in SEQ ID NO: 2. In some embodiments, the mutation is Y706D.

In some embodiments, the capsid protein of serotype AAV9 comprises a mutation in the N709 amino acid position in SEQ ID NO: 2. In some embodiments, the mutation is N709I. In some embodiments, the mutation is N709Y.

In some embodiments, the capsid protein of serotype AAV9 comprises one or more mutations in amino acid positions selected from the group consisting of: D327 and N329 in SEQ ID NO: 2. In some embodiments, the one or more mutations are D327T and N329D.

In some embodiments, the capsid protein of serotype AAV9 comprises one or more mutations in amino acid positions selected from the group consisting of: N552 and V553 in SEQ ID NO: 2. In some embodiments, the one or more mutations are N552A and V553A.

In some embodiments, the capsid protein of serotype AAV9 comprises one or more mutations in amino acid positions selected from the group consisting of: W595 and Q597 in SEQ ID NO: 2. In some embodiments, the one or more mutations are W595G and Q597S.

In some embodiments, the capsid protein of serotype AAV9 comprises one or more mutations in amino acid positions selected from the group consisting of: A656 and P658 in SEQ ID NO: 2. In some embodiments, the one or more mutations are: A656G and P658L.

In some embodiments, the capsid protein of serotype AAV9 comprises one or more mutations in amino acid positions selected from the group consisting of: Y706 and N709 in SEQ ID NO: 2. In some embodiments, the one or more mutations are: Y706D and N709I.

In some embodiments, the capsid protein of serotype AAV9 comprises one or more mutations in amino acid positions selected from the group consisting of: Q588 and T491 in SEQ ID NO: 2. In some embodiments, the one or more mutations are: Q588R and T491R.

In some embodiments, the one or more mutations are at amino acid positions T491, D556, N562, Q588, and Y706. In some embodiments, the one or more mutations are T491R, D556P, N562Y, Q588R, and Y706D.

In some embodiments, the rAAV9 particle further comprises one or more mutations at amino acid positions selected from the group consisting of: T582 and G594. In some embodiments, the one or more mutations are amino acid substitution(s) selected from the group consisting of: T582Q and G594E.

Each of the limitations of the invention can encompass various embodiments of the invention. It is, therefore, anticipated that each of the limitations of the invention involving any one element or combinations of elements can be included in each aspect of the invention. This disclosure is not limited in its application to the details of construction and the arrangement of components set forth in the following description or illustrated in the drawings. The invention is capable of other embodiments and of being practiced or of being carried out in various ways. Also, the phraseology and terminology used in this application is for the purpose of description and should not be regarded as limiting. The use of “including,” “comprising,” or “having,” “containing,” “involving,” and variations thereof, is meant to encompass the items listed thereafter and equivalents thereof as well as additional items. The term “a” or “an” refers to one or more of an entity.

### BRIEF DESCRIPTION OF DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIG. 1 shows 3D reconstruction maps of AAV9 (without Fab) and in complex with Fab1-(1-7)(patient 1) and Fab2-(1-7)(patient 2). These maps were used to determine Fab binding sites. The maps are radially colored according to distance to the capsid center (blue to red, as indicated by the scale bar). The positions of the icosahedral 2-, 3-, and 5-fold axes and the 2/5-fold wall are indicated above, and the binding sites for each Fab are given above each map. The estimated resolutions, determined at a FSC threshold value of 0.143, are shown below each map.

FIG. 2 shows an analysis of the cross-reactivity of the human antibodies. mAb-(1-7) (patient 1), mAb2-(1-7)(patient 2), and mAb3-(1-7)(patient 3) were probed against native capsids of AAV1-3, 5-9, and AAVrh.10 spotted on nitrocellulose membrane. Monoclonal antibody B1 served as an internal loading control with denatured capsids.

FIGS. 3A-3B show a summary of the generated AAV9 capsid variants. FIG. 3A shows the production efficiency and infectivity of each AAV9 capsid variant as a percentage, relative to wtAAV9. The results for the transgene expression in presence of the mAbs are color-coded according to the legend in FIG. 3B. Monoclonal antibody B1 served as an internal loading control with denatured capsids.

FIGS. 4A-4B show an AAV9 capsid variant (Q588R) escaping mAb1-1. FIG. 4A shows a native immune dot-blot with native wtAAV9 and AAV9-Q588R capsids spotted on nitrocellulose membrane probed with mAb1-1 and ADK9 (mouse monoclonal antibody). FIG. 4B shows a neutralization assay of wtAAV9 and AAV9-Q588R in variable concentrations of mAb1-1 and ADK9.

FIGS. 5A-5D show icosahedral reconstruction of the AAV9-Fab complexes. FIG. 5A shows surface density map of the cryo-reconstructed AAV9-Fab1-2 complex. The view is down the icosahedral 2-fold axis, the map is contoured at a sigma (o) threshold level of 2.0. The map is shaded according to radial distance from the particle center, as indicated by the scale bar. The icosahedral 2-, 3-, and 5-fold axes are indicated. The estimated resolution, determined at an FSC threshold value of 0.143, is shown below the map. The binding of the Fab on top of the 2/5-fold wall is shown as a close-up image in the center panel with the variable heavy (VH) and variable light (VL) chains labeled. To the right a representative stretch of amino acid residues modeled for the heavy chain of the Fab are shown inside the cryo-EM density map. The amino acid residues are labeled and shown as stick representations and colored according to atom type. FIG. 5B is a depiction of the AAV9-Fab2-7 complex as in (FIG. 5A) with the Fab binding around the 5-fold symmetry axis. FIG. 5C shows a surface density map of the AAV9-Fab3-4 complex as in (FIG. 5A). The Fab binds to the center of the 3-fold symmetry axis. Due to the imposed symmetry during the reconstruction process the density of the Fab is blurred and cannot be used to generate an atomic model. The illustration on the right gives a rationale for the blurring of a non-icosahedral Fab bound to an icosahedral capsid during icosahedral averaging. FIG. 5D is a depiction of the AAV9-Fab3-2 complex as in (FIG. 5C) with the Fab binding to the center of the 2-fold symmetry axis.

FIGS. 6A-6C show localized reconstruction of the AAV9-Fab complexes. FIG. 6A shows a surface density map of the icosahedral-reconstructed AAV9-Fab2-1 complex colored as in FIGS. 5A-5D. The icosahedral 2-, 3-, and 5-fold axes are indicated, and the estimated resolution is shown below the map. To the right a density map of the 2-fold region is shown derived from localized reconstruction with C2 symmetry relaxation. The variable/constant heavy (VH/CH) and variable/constant light (VL/CL) chains labeled. The 3-fold protrusions are indicated as black triangles. A representative stretch of amino acid residues modeled for the heavy chain of the Fab are shown inside the localized reconstructed map. The amino acid residues are labeled and shown as stick representations and colored according to atom type. FIG. 6B shows a depiction of the AAV9-Fab1-1 and FIG. 6C shows Fab1-6 complex as in (FIG. 6A). For the localized reconstruction of the 3-fold region C3 symmetry relaxation and the 5-fold region C5 symmetry relaxation were applied, respectively.

FIGS. 7A-7D show binding mode of the Fabs to the AAV9 capsid. FIG. 7A shows radial-colored surface representations of the AAV9 capsid are shown for double-trimers with the 2-fold symmetry axis in the center. The structures of the VH and VL chains are shown as ribbon diagrams. The 2-fold binding Fabs are classified into 4 groups based on their interaction to the AAV9 capsid. The Fabs belonging to the groups are listed below each surface representation. FIG. 7B shows surface representations of AAV9 capsid trimers showing the binding of Fab1-1 and Fab3-4. FIG. 7C shows AAV9 capsid pentamer showing the binding of Fab1-6 and Fab2-7. FIG. 7D shows surface representation as in FIG. 7A for Fab1-2.

FIGS. 8A-8D show the determination of the contacts of Fab2-1 to the AAV9 capsid. FIG. 8A shows modeled CDR1-3 of the heavy chain inside the cryo-EM map. The contacts to the AAV9 capsids including the distance of the interactions are listed below each model. FIG. 8B shows the fit of the AAV9 capsid and Fab2-1 models inside the cryo-EM map. FIG. 8C shows the modeled CDR1-3 of the light chain inside the cryo-EM map, as in (FIG. 8A). FIG. 8D shows exemplary contacts of the Fab to the AAV9 capsid.

FIGS. 9A-9D show contact frequency of the 2-fold binding Fabs to the AAV9 capsid. FIG. 9A shows the percentages of contacted capsid residues within VR-III, -IV, -V, B)-VI, -VII, and (FIG. 9C) VR-IX are shown for all 2-fold binding Fabs. FIG. 9D shows an AAV9 capsid surface representation with the binding frequency for any 2-fold binding Fabs, which is colored according to the bar on the right. FIG. 9A contains sequences corresponding to SEQ ID NOs: 3-5 from left to right. FIG. 9B contains sequences corresponding to SEQ ID NOs: 6-7 from left to right. FIG. 9C contains a sequence corresponding to SEQ ID NO: 8.

FIGS. 10A-10D show Fab induced structural rearrangement of the AAV9 capsid. FIG. 10A shows the binding of Fab1-2 to the AAV9 capsid leads to an alternative conformation of VR-I. The conformation in absence of the antibody and in presence of Fab1-2 are shown as ribbon diagrams with amino acid side chains. Movements of ≥3 Å between the indicated amino acids are indicated. FIG. 10B shows a depiction as in (FIG. 10A) for VR-II upon binding of Fab1-6, FIG. 10C shows VR-IV upon binding of Fab3-3, and FIG. 10D shows VR-VIII upon binding of Fab1-1.

FIGS. 11A-11C show the generation of an antibody escape variant. Native dot blots are shown using MAb B1 as a loading control and the 21 human Mabs against the capsids of wild-type AAV9, AAV5, and an engineered AAV9 capsid variants composed of 5 amino acid substitutions listed in the lower left corner. This capsid variant escapes 17 of the 21 human antibodies, with the exception of MAb1-2, 1-6, 2-7, and 3-4.

FIGS. 12A-12C show further improvement of the antibody escape variant. FIG. 12A shows native dot blots show that the introduction of the T582Q or G594E amino acid substitution to the 5-variant allows the escape from MAb3-4. FIG. 12B shows infectivity analyses show that both variants display enhanced transduction efficiencies over wild-type AAV9. FIG. 10C shows the transduction of the 5-variant is maintained in the presence of a pooled MAb solution which contained equal amounts of 17 different MAbs.

### DETAILED DESCRIPTION

Adeno-associated virus (AAV)-based therapeutic regimens are valuable options for treating diseases, such as, for example, spinal muscular atrophy (SMA). However, treatment with such therapeutic regimens can generate an undesirable immune response (e.g., neutralizing antibodies) in a subject against one or more aspects of the therapy, for example against the AAV vector used to deliver the therapeutic molecule. The presence of such neutralizing antibodies in a subject limits the use of additional AAV-based therapeutic regimens, as the subject will exhibit an immune response to AAV vectors of the same serotype backbone, or, in some embodiments, of a different serotype backbone (e.g., if the different serotype cross-reacts with the serotype against which the neutralizing antibodies were initially developed). Treating a subject having preexisting neutralizing antibodies that cross-react with a subsequent AAV-based therapeutic regimen can result in decreased effectiveness of the subsequent treatment and/or immunogenic effects to the subject.

Accordingly, aspects of the disclosure relate to a recombinant adeno-associated virus 9 (rAAV9) particle comprising a capsid protein comprising one or more mutations, wherein said mutations result in modulated reactivity to a neutralizing antibody, altered or unchanged production efficiency, and/or altered or unchanged infectivity, relative to a wild-type AAV9 particle. Aspects of the disclosure also relate to vectors and compositions comprising said rAAV9 particles.

In some embodiments, rAAV9 particles as described herein are useful for treating subjects with pre-existing neutralizing antibodies that react to or cross-react with AAV9, for example via the expression of therapeutic molecules (e.g., therapeutic proteins). Aspects of the disclosure thus relate to methods of delivering a protein of interest to a subject using a rAAV9 particle as described herein, or a composition or vector comprising the same. Aspects of the disclosure relate to vectors and kits useful in the preparation of rAAV particles.

### AAV9 Particles

Aspects of the present disclosure provide variant rAAV capsid proteins and particles having one or more mutations (e.g., amino acid substitutions, deletions, and/or insertions) relative to a wildtype AAV9 capsid protein or particle, and nucleic acids that encode the variant rAAV capsids. AAV9 is a human AAV serotype and belongs to AAV Clade F. AAV9 has enhanced transduction efficiency in certain tissues, including cardiac and skeletal muscle, liver and pancreatic tissue, and the eye, relative to other AAV serotypes, can cross the blood-brain barrier, and efficiently targets the central nervous system.

### Engineered AAV9 Particles

Disclosed herein are recombinant AAV9 (rAAV9) particles comprising a capsid protein comprising one or more mutations that result in modulated reactivity to neutralizing antibodies, altered or unchanged transduction efficiency, altered or unchanged production efficiency, and/or altered or unchanged infectivity, relative to a wild-type AAV9 particle.

In some embodiments, a variant recombinant AAV9 particle as disclosed herein comprises mutations (e.g., amino acid substitutions, deletions, and/or insertions) that result in altered transduction efficiency. In some embodiments, a variant recombinant AAV9 particle as disclosed herein comprises mutations that result in unchanged transduction efficiency. In some embodiments, mutations in the capsid protein of AAV9 particles result in both modulated reactivity to neutralizing antibodies and altered transduction efficiency. In some embodiments, mutations in the capsid protein of AAV9 particles result in both modulated reactivity to neutralizing antibodies and unchanged transduction efficiency. In some embodiments, mutations in the capsid protein of AAV9 particles result in decreased reactivity to neutralizing antibodies and unchanged transduction efficiency, relative to wild-type AAV9 particles. In some embodiments, mutations in the capsid protein of AAV9 particles result in decreased reactivity to neutralizing antibodies and increased transduction efficiency, relative to wild-type AAV9 particles. In some embodiments, mutations in the capsid protein of AAV9 particles result in decreased reactivity to neutralizing antibodies and unchanged transduction efficiency, relative to wild-type AAV9 particles. In some embodiments, mutations in the capsid protein of AAV9 particles results in decreased reactivity to neutralizing antibodies and decreased transduction efficiency, relative to wild-type AAV9 particles.

In some embodiments, a variant recombinant AAV9 particle as disclosed herein comprises mutations that result in altered production efficiency. In some embodiments, a variant recombinant AAV9 particle as disclosed herein comprises mutations that result in unchanged production efficiency. In some embodiments, mutations in the capsid protein of AAV9 particles result in both modulated reactivity to neutralizing antibodies and altered production efficiency. In some embodiments, mutations in the capsid protein of AAV9 particles result in both modulated reactivity to neutralizing antibodies and unchanged production efficiency. In some embodiments, mutations in the capsid protein of AAV9 particles results in decreased reactivity to neutralizing antibodies and unchanged production efficiency, relative to wild-type AAV9 particles. In some embodiments, mutations in the capsid protein of AAV9 particles results in decreased reactivity to neutralizing antibodies and increased production efficiency, relative to wild-type AAV9 particles. In some embodiments, mutations in the capsid protein of AAV9 particles results in decreased reactivity to neutralizing antibodies and decreased production efficiency, relative to wild-type AAV9 particles.

In some embodiments, a variant recombinant AAV9 particle as disclosed herein comprises mutations that result in altered infectivity. In some embodiments, a variant recombinant AAV9 particle as disclosed herein comprises mutations that result in unchanged infectivity. In some embodiments, mutations in the capsid protein of AAV9 particles result in both modulated reactivity to neutralizing antibodies and altered infectivity. In some embodiments, mutations in the capsid protein of AAV9 particles result in both modulated reactivity to neutralizing antibodies and unchanged infectivity. In some embodiments, mutations in the capsid protein of AAV9 particles results in decreased reactivity to neutralizing antibodies and unchanged infectivity, relative to wild-type AAV9 particles. In some embodiments, mutations in the capsid protein of AAV9 particles results in decreased reactivity to neutralizing antibodies and increased infectivity, relative to wild-type AAV9 particles. In some embodiments, mutations in the capsid protein of AAV9 particles results in decreased reactivity to neutralizing antibodies and decreased infectivity, relative to wild-type AAV9 particles.

The term “variant” or “mutant” as used herein means different from wild-type. As described herein, variant AAV9 particles are those that have an amino acid sequence that is different from the sequence of wild-type AAV9 particles (e.g., comprise one or more mutations relative to wild-type). A wild-type AAV9 particle may be one found in nature or a recombinant viral particle that is made in a laboratory setting for the purpose of testing phenotypes. A variant AAV9 particle as used herein is one that is engineered. As used herein, the term “engineered” is used interchangeably with the term “recombinant,” and refers to an AAV9 particle that has a capsid protein with an amino acid sequence that differs from wild-type AAV9.

In some embodiments, a recombinant AAV9 particle of the disclosure comprises a capsid protein comprising an amino acid sequence which differs from the amino acid sequence of a wild-type AAV9 capsid protein. In some embodiments, the difference in amino acid sequence is present in one or more capsid proteins (e.g., VP1, VP2, VP3, VP1 and VP2, VP1 and VP3, or VP1, VP2 and VP3). The AAV genome encodes overlapping sequences of the three capsid proteins, VP1, VP2 and VP3, which starts from one promoter. All three of the proteins are translated from one mRNA. After the mRNA is synthesized, it can be spliced in different ways, resulting in expression of the three proteins VP1, VP2 and VP3. The difference between the amino acid sequence between a “variant” AAV9 particle and a wild-type particle may be in one or more amino acids. For example, a variant AAV9 may contain 1 or 2, 3, 4, 5, 6, or 7 or more mutations relative to the amino acid sequence of wild-type AAV9. The nucleic acid encoding and the amino acid sequence of wild-type AAV9 capsid protein are provided as SEQ ID Nos: 1 and 2, respectively.

Nucleic acid sequence of the cap gene for wild-type AAV9
(SEQ ID NO: 1)
ATGGCTGCCGATGGTTATCTTCCAGATTGGCTCGAGGACAACCTTAGTGAAGGAATTCGCGAGT
GGTGGGCTTTGAAACCTGGAGCCCCTCAACCCAAGGCAAATCAACAACATCAAGACAACGCTCG
AGGTCTTGTGCTTCCGGGTTACAAATACCTTGGACCCGGCAACGGACTCGACAAGGGGGAGCCG
GTCAACGCAGCAGACGCGGCGGCCCTCGAGCACGACAAGGCCTACGACCAGCAGCTCAAGGCCG
GAGACAACCCGTACCTCAAGTACAACCACGCCGACGCCGAGTTCCAGGAGCGGCTCAAAGAAGA
TACGTCTTTTGGGGGCAACCTCGGGCGAGCAGTCTTCCAGGCCAAAAAGAGGCTTCTTGAACCT
CTTGGTCTGGTTGAGGAAGCGGCTAAGACGGCTCCTGGAAAGAAGAGGCCTGTAGAGCAGTCTC
CTCAGGAACCGGACTCCTCCGCGGGTATTGGCAAATCGGGTGCACAGCCCGCTAAAAAGAGACT
CAATTTCGGTCAGACTGGCGACACAGAGTCAGTCCCAGACCCTCAACCAATCGGAGAACCTCCC
GCAGCCCCCTCAGGTGTGGGATCTCTTACAATGGCTTCAGGTGGTGGCGCACCAGTGGCAGACA
ATAACGAAGGTGCCGATGGAGTGGGTAGTTCCTCGGGAAATTGGCATTGCGATTCCCAATGGCT
GGGGGACAGAGTCATCACCACCAGCACCCGAACCTGGGCCCTGCCCACCTACAACAATCACCTC
TACAAGCAAATCTCCAACAGCACATCTGGAGGATCTTCAAATGACAACGCCTACTTCGGCTACA
GCACCCCCTGGGGGTATTTTGACTTCAACAGATTCCACTGCCACTTCTCACCACGTGACTGGCA
GCGACTCATCAACAACAACTGGGGATTCCGGCCTAAGCGACTCAACTTCAAGCTCTTCAACATT
CAGGTCAAAGAGGTTACGGACAACAATGGAGTCAAGACCATCGCCAATAACCTTACCAGCACGG
TCCAGGTCTTCACGGACTCAGACTATCAGCTCCCGTACGTGCTCGGGTCGGCTCACGAGGGCTG
CCTCCCGCCGTTCCCAGCGGACGTTTTCATGATTCCTCAGTACGGGTATCTGACGCTTAATGAT
GGAAGCCAGGCCGTGGGTCGTTCGTCCTTTTACTGCCTGGAATATTTCCCGTCGCAAATGCTAA
GAACGGGTAACAACTTCCAGTTCAGCTACGAGTTTGAGAACGTACCTTTCCATAGCAGCTACGC
TCACAGCCAAAGCCTGGACCGACTAATGAATCCACTCATCGACCAATACTTGTACTATCTCTCA
AAGACTATTAACGGTTCTGGACAGAATCAACAAACGCTAAAATTCAGTGTGGCCGGACCCAGCA
ACATGGCTGTCCAGGGAAGAAACTACATACCTGGACCCAGCTACCGACAACAACGTGTCTCAAC
CACTGTGACTCAAAACAACAACAGCGAATTTGCTTGGCCTGGAGCTTCTTCTTGGGCTCTCAAT
GGACGTAATAGCTTGATGAATCCTGGACCTGCTATGGCCAGCCACAAAGAAGGAGAGGACCGTT
TCTTTCCTTTGTCTGGATCTTTAATTTTTGGCAAACAAGGAACTGGAAGAGACAACGTGGATGC
GGACAAAGTCATGATAACCAACGAAGAAGAAATTAAAACTACTAACCCGGTAGCAACGGAGTCC
TATGGACAAGTGGCCACAAACCACCAGAGTGCCCAAGCACAGGCGCAGACCGGCTGGGTTCAAA
ACCAAGGAATACTTCCGGGTATGGTTTGGCAGGACAGAGATGTGTACCTGCAAGGACCCATTTG
GGCCAAAATTCCTCACACGGACGGCAACTTTCACCCTTCTCCGCTGATGGGAGGGTTTGGAATG
AAGCACCCGCCTCCTCAGATCCTCATCAAAAACACACCTGTACCTGCGGATCCTCCAACGGCCT
TCAACAAGGACAAGCTGAACTCTTTCATCACCCAGTATTCTACTGGCCAAGTCAGCGTGGAGAT
CGAGTGGGAGCTGCAGAAGGAAAACAGCAAGCGCTGGAACCCGGAGATCCAGTACACTTCCAAC
TATTACAAGTCTAATAATGTTGAATTTGCTGTTAATACTGAAGGTGTATATAGTGAACCCCGCC
CCATTGGCACCAGATACCTGACTCGTAATCTGTAA
Amino acid sequence of the capsid protein for wild-type AAV9
(SEQ ID NO: 2)
MAADGYLPDWLEDNLSEGIREWWALKPGAPQPKANQQHQDNARGLVLPGYKYLGPGNGLDKGEP
VNAADAAALEHDKAYDQQLKAGDNPYLKYNHADAEFQERLKEDTSFGGNLGRAVEQAKKRLLEP
LGLVEEAAKTAPGKKRPVEQSPQEPDSSAGIGKSGAQPAKKRLNFGQTGDTESVPDPQPIGEPP
AAPSGVGSLTMASGGGAPVADNNEGADGVGSSSGNWHCDSQWLGDRVITTSTRTWALPTYNNHL
YKQISNSTSGGSSNDNAYFGYSTPWGYFDFNRFHCHFSPRDWQRLINNNWGFRPKRLNFKLFNI
QVKEVTDNNGVKTIANNLTSTVQVFTDSDYQLPYVLGSAHEGCLPPFPADVFMIPQYGYLTLND
GSQAVGRSSFYCLEYFPSQMLRTGNNFQFSYEFENVPFHSSYAHSQSLDRLMNPLIDQYLYYLS
KTINGSGQNQQTLKFSVAGPSNMAVQGRNYIPGPSYRQQRVSTTVTQNNNSEFAWPGASSWALN
GRNSLMNPGPAMASHKEGEDRFFPLSGSLIFGKQGTGRDNVDADKVMITNEEEIKTTNPVATES
YGQVATNHQSAQAQAQTGWVQNQGILPGMVWQDRDVYLQGPIWAKIPHTDGNFHPSPLMGGFGM
KHPPPQILIKNTPVPADPPTAFNKDKLNSFITQYSTGQVSVEIEWELQKENSKRWNPEIQYTSN
YYKSNNVEFAVNTEGVYSEPRPIGTRYLTRNL

In some embodiments, a mutation in a capsid protein of a variant recombinant AAV9 particle is an amino acid deletion. In some embodiments, a mutation is an amino acid substitution. In some embodiments, a substitution comprises an amino acid that is not present or conserved in one or more AAV particles of another serotype (e.g., AAV1, AAV2, AAV2.5, AAV3, AAV4, AAV5, AAV6, AAV7, AAV8, AAVrh.10, AAVrh.74, AAV10, AAV11, AAV12, AAV-DJ, PHP.eB, AvianAAV, or AAV13). In some embodiments, the other serotype may be an AAV isolated from rhesus macaque tissue. Gao, et al. (PNAS(2003); 100(10):6081-6086) discloses 37 capsid clones isolated from rhesus macaque tissues and is incorporated herein by reference in its entirety. In some embodiments, the other serotype may be an avian AAV. Avian AAV, also called “A3V”, has been isolated from birds, such as chickens (see e.g., Matsui, et al (PLOS One(2012): 7(11): e48730), which discloses the use of avian AAV to transduce neurons in chicken brains).

Sequences of other AAV serotypes are known and can be aligned with SEQ ID NO: 2 (wild-type AAV9 capsid protein) using techniques known in the art. In some embodiments, a substituted amino acid is not hydrophobic in nature. In some embodiments, mutations comprise substitutions with amino acid residues that are not found on AAV of other serotypes.

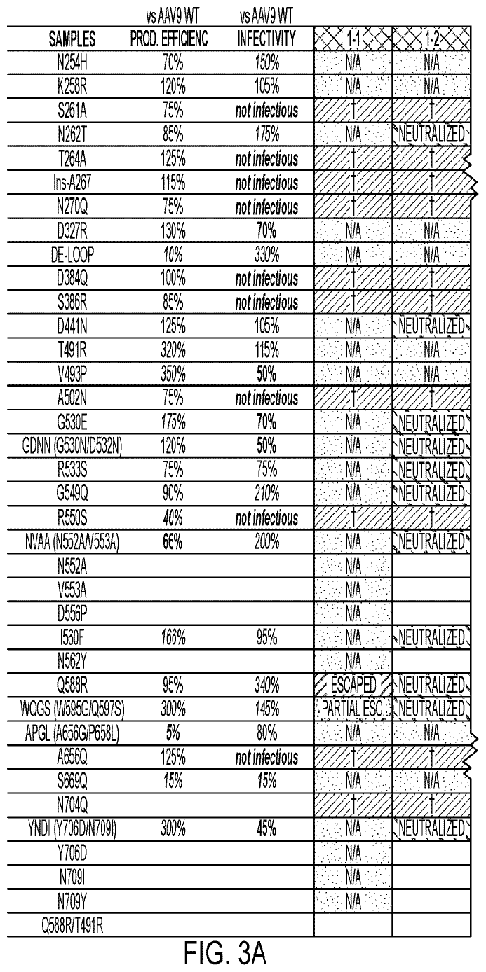

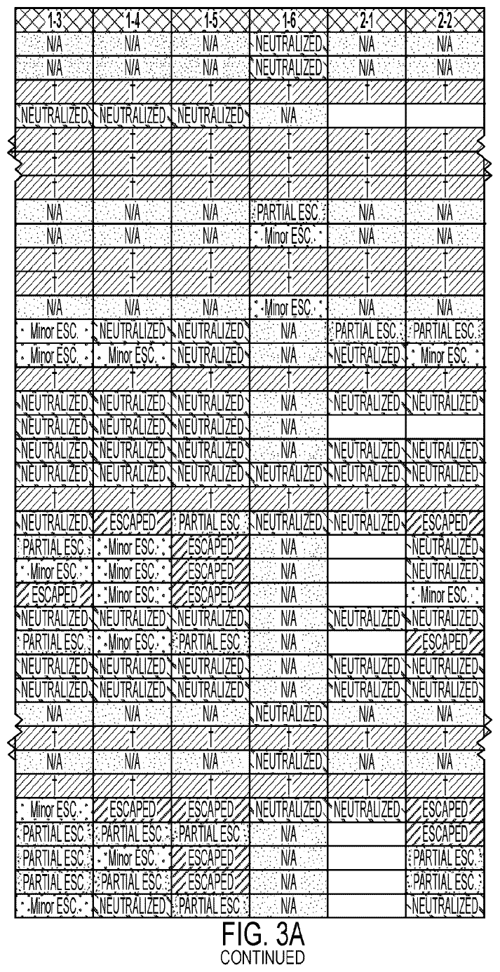

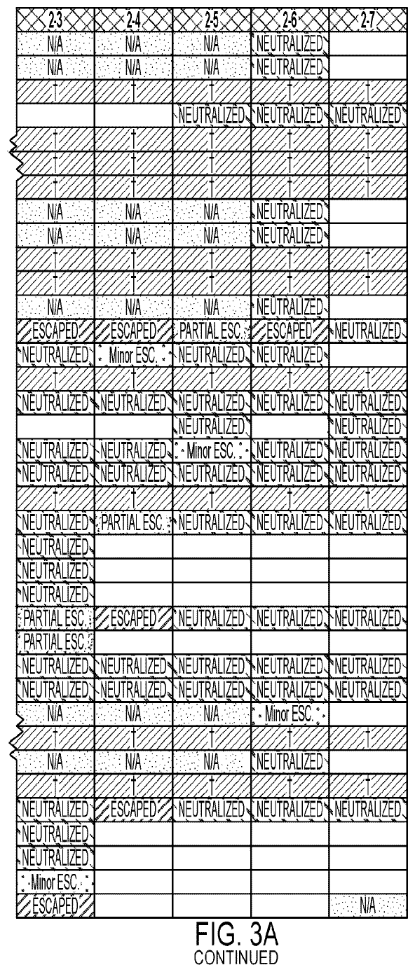

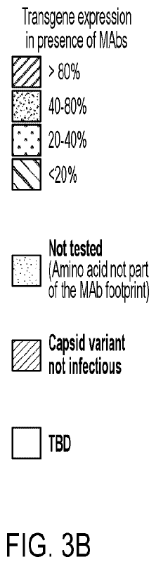

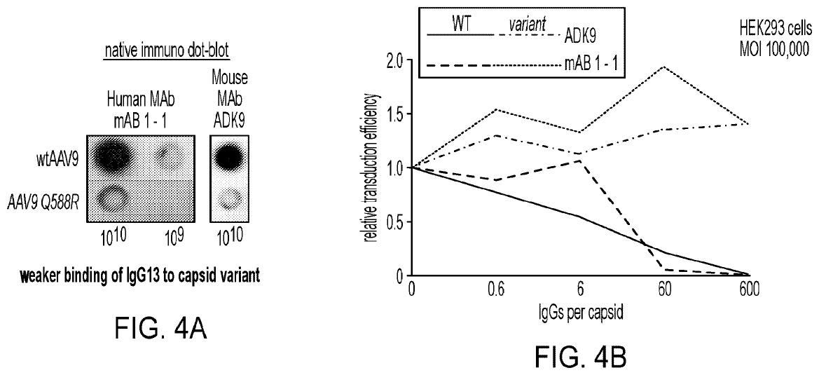

In some embodiments, more than one mutation is present on a recombinant or engineered AAV9 capsid (e.g., 2, 3, 4, 5, 6, or 7 or more mutations). In some embodiments, a recombinant or engineered AAV9 capsid comprises 1-10, 5-15, 10-20, 15-25, 20-30, 25-35, or 30-40 mutations (e.g., amino acid substitutions, deletions, and/or insertions). Non-limiting examples of AAV9 particles with multiple mutations are provided in FIGS. 3A and 3B. It is to be understood that any one mutation provided in this disclosure can be combined with any other mutation provided herein. It is also to be understood that AAV9 variants with multiple mutations provided herein are merely examples and are non-limiting. Any one of the mutations disclosed in any one variant AAV9 particle having multiple mutations may exist as a single mutation in an engineered AAV9 particle, or in combination with one or more of the other mutations disclosed here.

In some embodiments, a recombinant AAV9 particle as disclosed herein comprises one or more mutations at positions selected from the group consisting of: N254, K258, S261, N262, T264, S265, G266, G267, Ins-A267, N270, D327, N329, D384, S386, D441, T491, V493, T494, A502, G530, D532, R533, G549, R550, D551, N552, V553, D556, 1560, N562, T582, Q588, A589, G594, W595, Q597, N598, A656, P658, N663, N668, S669, N704, Y706, N709, and T717.

In some embodiments, the one or more mutations comprise a mutation at position N254. In some embodiments, the one or more mutations comprise a mutation at position K258. In some embodiments, the one or more mutations comprise a mutation at position S261. In some embodiments, the one or more mutations comprise a mutation at position N262. In some embodiments, the one or more mutations comprise a mutation at position T264. In some embodiments, the one or more mutations comprise a mutation at position S265. In some embodiments, the one or more mutations comprise a mutation at position G266. In some embodiments, the one or more mutations comprise a mutation at position G267. In some embodiments, the one or more mutations comprise an insertion, wherein an A amino acid is inserted before position G267 (e.g., the G amino acid located at position 267 in SEQ ID NO: 2), such that the inserted A is at position 267 (e.g., the A amino acid is located at position 267 in SEQ ID NO: 2) and G267 is shifted to G268 (e.g., the G amino acid located at position 268 in SEQ ID NO: 2). This mutation is referred to herein as “Ins-A267.” In some embodiments, the one or more mutations comprise a mutation at position N270. In some embodiments, the one or more mutations comprise a mutation at position D327. In some embodiments, the one or more mutations comprise a mutation at position N329. In some embodiments, the one or more mutations comprise a mutation at position D384. In some embodiments, the one or more mutations comprise a mutation at position S386. In some embodiments, the one or more mutations comprise a mutation at position D441. In some embodiments, the one or more mutations comprise a mutation at position T491. In some embodiments, the one or more mutations comprise a mutation at position V493. In some embodiments, the one or more mutations comprise a mutation at position T494. In some embodiments, the one or more mutations comprise a mutation at position A502. In some embodiments, the one or more mutations comprise a mutation at position G530. In some embodiments, the one or more mutations comprise a mutation at position D532. In some embodiments, the one or more mutations comprise a mutation at position R533. In some embodiments, the one or more mutations comprise a mutation at position G549. In some embodiments, the one or more mutations comprise a mutation at position R550. In some embodiments, the one or more mutations comprise a mutation at position D551. In some embodiments, the one or more mutations comprise a mutation at position N552. In some embodiments, the one or more mutations comprise a mutation at position V553. In some embodiments, the one or more mutations comprise a mutation at position D556. In some embodiments, the one or more mutations comprise a mutation at position 1560. In some embodiments, the one or more mutations comprise a mutation at position N562. In some embodiments, the one or more mutations comprise a mutation at position T582. In some embodiments, the one or more mutations comprise a mutation at position Q588. In some embodiments, the one or more mutations comprise a mutation at position A589. In some embodiments, the one or more mutations comprise a mutation at position G594. In some embodiments, the one or more mutations comprise a mutation at position W595. In some embodiments, the one or more mutations comprise a mutation at position Q597. In some embodiments, the one or more mutations comprise a mutation at position N598. In some embodiments, the one or more mutations comprise a mutation at position A656. In some embodiments, the one or more mutations comprise a mutation at position P658. In some embodiments, the one or more mutations comprise a mutation at position N663. In some embodiments, the one or more mutations comprise a mutation at position N668. In some embodiments, the one or more mutations comprise a mutation at position S669. In some embodiments, the one or more mutations comprise a mutation at position N704. In some embodiments, the one or more mutations comprise a mutation at position Y706. In some embodiments, the one or more mutations comprise a mutation at position N709. In some embodiments, the one or more mutations comprise a mutation at position T717.

In some embodiments, a recombinant AAV9 particle as disclosed herein comprises one or more mutations (e.g., amino acid substitution(s)) selected from the group consisting of: N254H, N254Q, N254Y, K258R, S261A, N262T, T264A, S265E, G266S, G267S, Ins-A267, N270Q, D327R, D327T, N329D, D384Q, S386R, D441N, T491R, V493P, T494Q, A502N, G530E, G530N, D532N, R533S, G549Q, R550S, D551A, N552A, V553A, D556P, I560F, N562Y, T582Q, Q588R, A589N, G594E, W595G, Q597S, N598L, A656G, A656S, A656Q, P658L, N663Q, N668S, S669Q, N704Q, Y706D, N709I, N709Y, and T717Q.

In some embodiments, the one or more mutations comprise N254H. In some embodiments, the one or more mutations comprise N254Q. In some embodiments, the one or more mutations comprise N254Y. In some embodiments, the one or more mutations comprise K258R. In some embodiments, the one or more mutations comprise S261A. In some embodiments, the one or more mutations comprise N262T. In some embodiments, the one or more mutations comprise T264A. In some embodiments, the one or more mutations comprise S265E. In some embodiments, the one or more mutations comprise G266S. In some embodiments, the one or more mutations comprise G267S. In some embodiments, the one or more mutations comprise Ins-A267. In some embodiments, the one or more mutations comprise N270Q. In some embodiments, the one or more mutations comprise D327R. In some embodiments, the one or more mutations comprise D327T. In some embodiments, the one or more mutations comprise N329D. In some embodiments, the one or more mutations comprise D384Q. In some embodiments, the one or more mutations comprise S386R. In some embodiments, the one or more mutations comprise D327T. In some embodiments, the one or more mutations comprise N329D. In some embodiments, the one or more mutations comprise D441N. In some embodiments, the one or more mutations comprise T491R. In some embodiments, the one or more mutations comprise V493P. In some embodiments, the one or more mutations comprise T494Q. In some embodiments, the one or more mutations comprise A502N. In some embodiments, the one or more mutations comprise G530E. In some embodiments, the one or more mutations comprise G530N. In some embodiments, the one or more mutations comprise D532N. In some embodiments, the one or more mutations comprise R533S. In some embodiments, the one or more mutations comprise G549Q. In some embodiments, the one or more mutations comprise R550S. In some embodiments, the one or more mutations comprise D551A. In some embodiments, the one or more mutations comprise N552A. In some embodiments, the one or more mutations comprise V553A. In some embodiments, the one or more mutations comprise D556P. In some embodiments, the one or more mutations comprise I560F. In some embodiments, the one or more mutations comprise N562Y. In some embodiments, the one or more mutations comprise T582Q. In some embodiments, the one or more mutations comprise Q588R. In some embodiments, the one or more mutations comprise A589N. In some embodiments, the one or more mutations comprise G594E. In some embodiments, the one or more mutations comprise W595G. In some embodiments, the one or more mutations comprise Q597S. In some embodiments, the one or more mutations comprise N598L. In some embodiments, the one or more mutations comprise A656G. In some embodiments, the one or more mutations comprise A656S. In some embodiments, the one or more mutations comprise A656Q. In some embodiments, the one or more mutations comprise P658L. In some embodiments, the one or more mutations comprise N663Q. In some embodiments, the one or more mutations comprise N668S. In some embodiments, the one or more mutations comprise S669Q. In some embodiments, the one or more mutations comprise N704Q. In some embodiments, the one or more mutations comprise Y706D. In some embodiments, the one or more mutations comprise N709I. In some embodiments, the one or more mutations comprise N709Y. In some embodiments, the one or more mutations comprise T717Q.

In some embodiments, a recombinant AAV9 particle as disclosed herein may contain more than one of any of the mutations described above. In some embodiments, a recombinant AAV9 particle as disclosed herein comprises the mutations D327T and N329D. In some embodiments, a recombinant AAV9 particle as disclosed herein comprises the mutations G530N and D532N. In some embodiments, a recombinant AAV9 particle as disclosed herein comprises the mutations N552A and V553A. In some embodiments, a recombinant AAV9 particle as disclosed herein comprises the mutations Q588R and T491R. In some embodiments, a recombinant AAV9 particle as disclosed herein comprises the mutations W595G and Q597S. In some embodiments, a recombinant AAV9 particle as disclosed herein comprises the mutations, A656G and P658L. In some embodiments, a recombinant AAV9 particle as disclosed herein comprises the mutations Y706D and N709I.

FIGS. 3A and 3B list some non-limiting examples of variant AAV9 particles and their observed phenotypes.

### Cross Reactivity of AAV9 to Neutralizing Antibodies

As will be understood, neutralizing antibodies are developed in a subject in response to an antigen, for example an AAV. Once developed, neutralizing antibodies target the antigen, and neutralize the biological effect of said antigen (either in whole or in part). In some embodiments, cross-reactivity may occur, wherein neutralizing antibodies generated against a first antigen (e.g., AAV8) may also target and neutralize (either in whole or in part) a second antigen (e.g., AAV9). For example, once exposed to a first AAV serotype (e.g., AAV8), a subject may develop antibodies that neutralize both that first AAV serotype and at least one other AAV serotype (e.g., AAV9).

In some embodiments, the presence of such cross-reactive neutralizing antibodies reduces or eliminates the effectiveness of AAV-based therapeutic regimens. In some embodiments, treatment with a therapeutic regimen comprising an AAV particle against which a subject has developed a neutralizing antibody can cause decreased efficacy of the therapy (and/or subsequent AAV-based therapies) and may in some embodiments have detrimental immunogenic effects in the subject.

In some embodiments, a neutralizing antibody to which an engineered variant AAV9 particle shows modulated reactivity relative to a wild-type AAV9 particle may be any neutralizing antibody that reacts to any AAV particle. In some embodiments, a neutralizing antibody is against AAV9. In some embodiments, a neutralizing antibody is against an AAV particle of another serotype (e.g., AAV1, AAV2, AAV2.5, AAV3, AAV4, AAV5, AAV6, AAV7, AAV8, AAVrh. 10, AAVrh.74, AAV10, AAV11, AAV12, AAV-DJ, PHP.eB, AvianAAV, or AAV13). In some embodiments, the other serotype may be an AAV isolated from rhesus macaque tissue. In some embodiments, the other serotype is AAV1, AAV2, AAV2.5, AAV3, AAV4, AAV5, AAV6, AAV7, AAV8, AAVrh. 10, AAVrh.74, AAV10, AAV11, AAV12, AAV-DJ, PHP.eB, AvianAAV, or AAV13.

In some embodiments, a neutralizing antibody to which an engineered variant AAV9 particle shows modulated reactivity relative to wild-type AAV9 reacts to AAVs of more than one serotype (e.g., an antibody reacting to both AAV8 and AAV3). In some embodiments, an AAV other than AAV9 is a hybrid AAV and comprises sequences from more than one AAV serotype.

In some embodiments, the neutralizing antibody is or comprises an immunoglobulin. As will be understood, an immunoglobulin is a heterodimeric protein comprising two heavy (H) and two light (L) chains (see, e.g., Schroeder & Cavacini, J Allergy Clin Immunol (2010); 125 (202): S41-S52). Immunoglobulins (e.g., antibodies) are characterized according to their heavy chain constant region. They may be classified as Immunoglobulin G (IgG), Immunoglobulin M (IgM), Immunoglobulin A (IgA), Immunoglobulin D (IgD), or Immunoglobulin E (IgE). In some embodiments, the neutralizing antibody is or comprises IgG, IgM, IgA, IgD, or IgE. In some embodiments, the neutralizing antibody is or comprises IgG. In some embodiments, the neutralizing antibody is or comprises IgG1. In some embodiments, the neutralizing antibody is or comprises IgG2. In some embodiments, the neutralizing antibody is or comprises IgG3. In some embodiments, the neutralizing antibody is or comprises IgG4. In some embodiments, the neutralizing antibody is or comprises IgM. In some embodiments, the neutralizing antibody is or comprises IgA. In some embodiments, the neutralizing antibody is or comprises IgA1. In some embodiments, the neutralizing antibody is or comprises IgA2. In some embodiments, the antibody is or comprises IgD. In some embodiments, the neutralizing antibody is or comprises IgE.

As will be understood, neutralizing antibodies may be developed over time in a subject, in response to antigens. For example, over 30% of the human population has neutralizing antibodies against AAV (e.g., AAV9, or an AAV serotype that cross-reacts with AAV9 (e.g., AAV8, AAVrh.10); see, e.g., Weber (Front Immunol (2021); 12:658399)). In some embodiments, the neutralizing antibody is a pre-existing neutralizing antibody against AAV. In some embodiments, the neutralizing antibody is generated in a subject in response to administration of an AAV vector. In some embodiments, the neutralizing antibody is generated in a subject in response to administration of an AAV9 vector. In some embodiments, the neutralizing antibody is generated in a subject in response to administration of an AAV vector having a serotype that cross-reacts with AAV9 (e.g., AAV8, AAVrh.10). In some embodiments, the neutralizing antibody is a pre-existing neutralizing antibody against AAV9. In some embodiments, the neutralizing antibody is a pre-existing neutralizing antibody against an AAV having a serotype that cross-reacts with AAV9 (e.g., AAV8, AAVrh.10).

In some embodiments, the neutralizing antibody is isolated from the subject. As will be understood, antibodies may in some embodiments be isolated from a subject's serum or peripheral blood mononuclear cells (PBMC)(see, e.g., Calcedo, et al (J Virol(2009)83; 6:2623-2631). As discussed above, neutralizing antibodies may be developed over time in a subject in response to an antigen. Different subjects may develop antibodies that recognize different portions of an antigen. As will be understood, and without wishing to be bound by theory, isolating neutralizing antibodies from a specific subject allows for screening of AAV9 capsids that will not generate a deleterious immunogenic response in that specific subject.

In some embodiments, the neutralizing antibody comprises one or more Fragment antigen-binding (Fab) region(s). As will be understood, wild-type antibodies comprise Fab regions and crystallizable fragment (Fc) regions. Fab regions are antibody regions that bind to an antigen. Fc regions comprise two variable regions and two constant regions. The two variable regions are the heavy variable region (V_{H}) and the light variable (V_{L}) region. The two constant domains are the heavy chain first constant region (C_{H1}) and the light chain constant region (CL). In some embodiments, the neutralizing antibody comprises one or more Fab region(s) which binds AAV9. As will be understood, Fab regions bind an antigen through interactions between the antigen and the Fab's complementary determining regions (CDRs), also called hypervariable loops. The V_{H} and V_{L} regions of a Fab each contain three CDRs. The CDRs form a binding pocket which recognizes and binds to a portion of an antigen known as an epitope (see, e.g., Chiu, et al., Antibodies (2019)8; 4:55). In some embodiments, the neutralizing antibody comprises one Fab region which binds AAV9. As will be understood, antibody fragments may be generated which comprise only one Fab region. In some embodiments, the neutralizing antibody is an antibody fragment comprising only one Fab. Antibodies with two Fabs that each bind a different antigen may also be generated. These antibodies are known as bivalent antibodies. In some embodiments, the neutralizing antibody is or comprises a bivalent antibody. In some embodiments, the neutralizing antibody comprises more than one Fab region which binds AAV9. As discussed above, wild-type antibodies generally comprise two Fab regions which bind the same epitope. In some embodiments, the neutralizing antibody comprises two Fab regions which bind AAV9. In some embodiments, the neutralizing antibody comprises two Fab regions which bind the same AAV9 epitope. In some embodiments, the neutralizing antibody comprises two Fab regions which bind two different AAV9 epitopes.

In some embodiments, a mutation in a capsid protein of any one of the recombinant AAV9 particles disclosed herein results in decreased reactivity to neutralizing antibodies relative to a wild-type AAV9 particle. In some embodiments, reactivity to neutralizing antibodies is decreased by 5-100% (e.g., 5-10, 10-20, 20-40, 40-60, 60-80, 80-90, 90-95, 95-98, 95-99, 95-99.9, 100, 10-100, 30-100, 60-100, 70-100 or 80-100%) relative to wild-type AAV9 particles. In some embodiments, reactivity to neutralizing antibodies is decreased by 10-90%, 20-100%, 30-90%, 40-100%, 50-100%, 50-75%, 75-100%, or 25-50%, relative to wild-type AAV9 particles. In some embodiments, reactivity to neutralizing antibodies is decreased 1.5-fold, 2-fold, 2.5-fold, 3-fold, 4-fold, 5-fold, 7.5-fold, or 10-fold, relative to wild-type AAV9 particles. In some embodiments, reactivity to neutralizing antibodies is decreased 1-10 fold, 2-20-fold, 10-20-fold, 5-25-fold, 20-100-fold, relative to wild-type AAV9 particles.

Non-limiting examples of AAV9 capsid protein mutations that result in antibody escape and/or decreased reactivity to neutralizing antibodies are provided in FIGS. 3A and 3B.

Reactivity of a recombinant AAV9 particle to a neutralizing antibody can be measured in one of numerous methods known in the art for measuring binding of a virus particle to a protein (e.g., dot blotting, surface plasmon resonance or biolayer interferometry). Neutralization of virus infectivity by antibodies is described by Klasse (Adv Biol.(2014); 2014:157895). Without intending to be bound by theory, antibodies can neutralize virus infectivity by interfering with essential steps of the viral life cycle. Virus infectivity can be neutralized before or after viral entry, depending on the virus. In an embodiment, for example, a neutralizing antibody may sterically block a viral entry protein from binding to its target receptor on a cell surface. In an embodiment, a neutralizing antibody interferes with viral life cycle steps after entry into the cell, such as exit from an endosome or extrusion of a genome.

### Production Efficiency, Transduction Efficiency, and Infectivity

Here, “production efficiency” means the ability of an engineered strain to produce functional rAAV particles, relative to wild-type AAV9. See, e.g., Mietzsch, et al (J Vir(2012) 95(19):JVI0077321). The term “infectivity” as used herein refers to the entry of an AAV particle into a cell, and is equivalent to “infection” of a cell.

In some embodiments, one or more mutations in a capsid protein of an AAV9 particle results in altered (e.g., increased or decreased) production efficiency, relative to a wild type AAV9 particle.

In some embodiments, the production efficiency of a variant AAV9 particle as described herein is increased relative to the production efficiency of a wild-type AAV9 particle. In some embodiments, the production efficiency of a variant AAV9 particle is increased 1.2-500 fold (e.g., 1.2-2, 1.5-3, 3-5, 5-10, 10-100, 100-250, 150-300, 250-350, 200-400, or 200-500 fold) relative to the production efficiency of a wild-type AAV9 particle for the same type of cell. In some embodiments, the production efficiency of a variant AAV9 particle is increased by 5-400% (e.g., 5-10, 10-30, 20-50, 50-100, 5-60, 100-200, 150-250, 200-400, 250-350, 300-400%) relative to the production efficiency of a wild-type AAV9 particle for the same type of cell.

In some embodiments, the production efficiency of a variant AAV9 particle is decreased relative to the production efficiency of a wild-type AAV9 particle. In some embodiments, the production efficiency of a variant AAV9 particle is decreased 1.2-500 fold (e.g., 1.2-500 fold (e.g., 1.2-2, 1.5-3, 3-5, 5-10, 10-100, 100-250, 150-300, 250-350, 200-400, or 200-500 fold) relative to the production efficiency of a wild-type AAV9 particle for the same type of cell. In some embodiments, the production efficiency of a variant AAV9 particle is decreased by 5-100% (e.g., 5-100, 5-10, 10-30, 20-50, 20-70, 50-100, 5-60, 20-80 or 80-100%) relative to the production efficiency of a wild-type AAV9 particle for the same type of cell.

In some embodiments, a mutation in a AAV9 capsid protein does not alter the production efficiency of the variant AAV9 particle harboring the mutation relative to the production efficiency of a wild-type AAV9 particle (e.g., the production efficiency of the recombinant AAV9 particle is “unchanged” or relatively unchanged, relative to a wild type AAV9 particle). As used herein, “unchanged” means unchanged or relatively unchanged (e.g., the production efficiency of the variant AAV particle is within 1%, 2%, or 3% of the production efficiency of a wild-type AAV9 particle).

In some embodiments, one or more mutations in a capsid protein of an AAV9 particle results in altered transduction efficiency. The term “transduction” as used herein refers to the entry of an AAV particle into a cell, and is equivalent to “infection” of a cell.

In some embodiments, the transduction efficiency is increased compared to the transduction efficiency of a wild-type AAV9 particle. In some embodiments, the transduction efficiency of a variant AAV9 particle is increased 1.2-500 fold (e.g., 1.2-2, 1.5-3, 3-5, 5-10, 10-100, 100-250 or 200-500 fold) compared to the transduction efficiency of a wild-type AAV9 particle for the same type of cell. In some embodiments, the transduction efficiency of a variant AAV9 particle is increased by 150-400% (e.g., 150-200, 150-300, 200-350, 250-400, 150-360 or 300-400%) compared to the transduction efficiency of a wild-type AAV9 particle for the same type of cell.

In some embodiments, a mutation in a AAV9 capsid protein does not alter the transduction efficiency of the variant AAV9 particle harboring the mutation, relative to the transduction efficiency of a wild-type AAV9 particle (e.g., the transduction efficiency of the recombinant AAV9 particle is “unchanged” or relatively unchanged, relative to a wild type AAV9 particle). As used herein, “unchanged” means unchanged or relatively unchanged (e.g., the transduction efficiency of the variant AAV particle is within 1%, 2%, or 3% of the transduction efficiency of a wild-type AAV9 particle).

In some embodiments, one or more mutations in a capsid protein of an AAV9 particle results in altered (e.g., increased or decreased) infectivity, relative to a wild type AAV9 particle.

In some embodiments, the infectivity of a variant AAV9 particle as described herein is increased relative to the infectivity of a wild-type AAV9 particle. In some embodiments, the infectivity of a variant AAV9 particle is increased 1.2-500 fold (e.g., 1.2-500 fold (e.g., 1.2-2, 1.5-3, 3-5, 5-10, 10-100, 100-250, 150-300, 250-350, 200-400, or 200-500 fold) relative to the infectivity of a wild-type AAV9 particle for the same type of cell. In some embodiments, the infectivity of a variant AAV9 particle is increased by 5-200% (e.g., 5-10, 10-30, 20-50, 50-100, 5-60, 100-200, 150-250, 200-400, 250-350, 300-400%) relative to the infectivity of a wild-type AAV9 particle for the same type of cell.

In some embodiments, the infectivity of a variant AAV9 particle is decreased, relative to the infectivity of a wild-type AAV9 particle. In some embodiments, the infectivity of a variant AAV9 particle is decreased 1.2-500 fold (e.g., 1.2-2, 1.5-3, 3-5, 5-10, 10-100, 100-250, 150-300, 250-350, 200-400, or 200-500 fold), relative to the infectivity of a wild-type AAV9 particle for the same type of cell. In some embodiments, the infectivity of a variant AAV9 particle is decreased by 5-100% (e.g., 5-100, 5-10, 10-30, 20-50, 20-70, 50-100, 5-60, 20-80 or 80-100%), relative to the infectivity of a wild-type AAV9 particle for the same type of cell. In some embodiments, at least about 50% of wild-type infectivity is retained in the variant AAV9 particle (e.g., 40-42%, 41-43%, 42-44%, 43-45%, 44-46%, 45-47%, 46-48%, 47-49%, 48-50%, 49-51%, 50-52%, 51-53%, 52-54%, 53-55%, 54-56%, 55-57%, 56-58%, 57-59%, or 58-60%).

In some embodiments, a mutation in a AAV9 capsid protein does not alter the infectivity of the variant AAV9 particle harboring the mutation relative to the infectivity of a wild-type AAV9 particle (e.g., the infectivity of the recombinant AAV9 particle is “unchanged” or relatively unchanged, relative to a wild type AAV9 particle). As used herein, “unchanged” means unchanged or relatively unchanged (e.g., the infectivity of the variant AAV particle is within 1%, 2%, or 3% of the infectivity of a wild-type AAV9 particle).

### Recombinant AAV Particles Comprising Recombinant Nucleic Acids

Aspects of the invention relate to recombinant AAV particles, as described herein, comprising recombinant nucleic acids. Such rAAV particles may be useful, for example, in certain therapeutic applications. In some embodiments, any one of the recombinant AAV9 particles disclosed herein comprises a recombinant nucleic acid (e.g., arAAV genome). In some embodiments, the recombinant nucleic acid comprises a transgene. In some embodiments, the transgene comprises a gene of interest. In some embodiments, the transgene is operably linked or operably connected to a promoter. Accordingly, in some embodiments, a rAAV9 particle comprises a transgene encoding a gene of interest, wherein the transgene is encapsidated by the viral capsid. In some embodiments, a recombinant AAV9 particle as disclosed herein is an empty capsid.

In some embodiments, a gene of interest encodes a therapeutic protein. A therapeutic protein may be an antibody, a peptibody, a growth factor, a clotting factor, a hormone, a membrane protein, a cytokine, a chemokine, an activating or inhibitory peptide acting on cell surface receptors or ion channels, a cell-permeant peptide targeting intracellular processes, a thrombolytic, an enzyme, a bone morphogenetic proteins, a nuclease or other protein used for gene editing, an Fc-fusion protein, or an anticoagulant.

In some embodiments, the gene of interest comprises a survival motor neuron (SMN) gene, such as SMN1 or SMN2. In some embodiments, the therapeutic protein is a survival motor neuron (SMN) protein. In some embodiments, the SMN protein is SMN1 or SMN2. In some embodiments, the gene of interest comprises a major facilitator superfamily (MFS) member gene, such as major facilitator superfamily domain containing 8 (MFSD8). In some embodiments, the therapeutic protein is a major facilitator superfamily family (MFS) protein. In some embodiments, the therapeutic protein is major facilitator superfamily domain containing 8 (MFSD8). In some embodiments, the gene of interest comprises an asparoacylase (ASPA) gene. In some embodiments, the therapeutic protein is an asparoacylase (ASPA) protein. In some embodiments, the gene of interest is an alpha-glucosidase (GAA) gene. In some embodiments, the therapeutic protein is an alpha-glucosidase (GAA) protein. In some embodiments, the gene is a lysosomal acid β-galactosidase (GLB1) gene. In some embodiments, the therapeutic protein is a lysosomal acid β-galactosidase (GLB1) protein.

In some embodiments, a gene of interest encodes a detectable molecule. In some embodiments, a detectable molecule is a fluorescent protein, a bioluminescent protein, or a protein that provides color (e.g., β-galactosidase, β-lactamases, β-glucuronidase and spheriodenone), or a fragment thereof. In some embodiments, a detectable molecule is a fluorescent, bioluminescent or enzymatic protein or functional peptide or functional polypeptide thereof.

In some embodiments, fluorescent protein is a blue fluorescent protein, a cyan fluorescent protein, a green fluorescent protein, a yellow fluorescent protein, an orange fluorescent protein, a red fluorescent protein, or functional peptides or polypeptides thereof. A blue fluorescent protein may be azurite, EBFP, EBFP2, mTagBFP, or Y66H. A cyan fluorescent protein may be ECFP, AmCyan1, Cerulean, CyPet, mECFP, Midori-ishi Cyan, mTFP1, or TagCFP. A Green fluorescent protein may be AcGFP, Azami Green, EGFP, Emarald, GFP or a mutated form of GFP (e.g., GFP-S65T, mWasabi, Stemmer, Superfolder GFP, TagGFP, TurboGFP, and ZsGreen). A yellow fluorescent protein may be EYFP, mBanana, mCitrine, PhiYFp, Tag YFP, Topaz, Venus, YPet, or ZsYellow1. An orange fluorescent protein may be DsRed, RFP, DsRed2, DsRed-Express, Ds-Red-monomer, Tomato, tdTomato, Kusabira Orange, mKO2, mOrange, mOrange2, mTangerine, TagRFP, or TagRFP-T. A red fluorescent protein may be AQ142, AsRed2, dKeima-Tandem, HcRed1, tHcRed, Jred, mApple, mCherry, mPlum, mRaspberry, mRFP1, mRuby or mStrawberry.

In some embodiments, a detectable molecule is a bioluminescent protein, or functional peptide or polypeptide thereof. Non-limiting examples of bioluminescent proteins are firefly luciferase, click-beetle luciferase, Renilla luciferase, or luciferase from Oplophorus gracilirostris.

In some embodiments, a detectable molecule may be any polypeptide or protein that can be detected using methods known in the art. Non-limiting methods of detection are fluorescence imaging, luminescent imaging and bright field imaging.

In some embodiments, a nucleic acid is provided, the nucleic acid comprising an expression construct containing a promoter operably linked to a coding sequence of a gene of interest. In some embodiments, a promoter is a natural promoter. In some embodiments, a promoter can be a truncated natural promoter. In some embodiments, a promoter can include an enhancer and/or basal promoter elements from a natural promoter. In some embodiments, a promoter can be or include elements from a CMV, a chicken beta actin, a desmin, or any other suitable promoter or combination thereof. In some embodiments, a promoter can be an engineered promoter. In some embodiments, a promoter is transcriptionally active in host cells. In some embodiments, a promoter is less than 1.6 kb in length, less than 1.5 kb in length, less than 1.4 kb in length, less than 1.3 kb in length, less than 1.2 kb in length, less than 1.1 kb in length, less than 1 kb in length, or less than 900 kb in length.

A promoter is “operably linked” to a nucleotide sequence when the promoter sequence controls and/or regulates the transcription of the nucleotide sequence. A promoter may be a constitutive promoter, tissue-specific promoter, an inducible promoter, or a synthetic promoter.

For example, constitutive promoters of different strengths can be used. A nucleic acid vector described herein may include one or more constitutive promoters, such as viral promoters or promoters from mammalian genes that are generally active in promoting transcription. Non-limiting examples of constitutive viral promoters include the Herpes Simplex virus (HSV), thymidine kinase (TK), Rous Sarcoma Virus (RSV), Simian Virus 40 (SV40), Mouse Mammary Tumor Virus (MMTV), Ad ElA cytomegalovirus (CMV) promoters. Non-limiting examples of constitutive mammalian promoters include various housekeeping gene promoters, as exemplified by the β-actin promoter (e.g., chicken β-actin promoter) and human elongation factor-1 α (EF-1α) promoter. In some embodiments, chimeric viral/mammalian promoters may include a chimeric CMV/chicken beta actin (CBA, CB or CAG) promoters.

Inducible promoters and/or regulatory elements may also be contemplated for achieving appropriate expression levels of the protein or polypeptide of interest. Non-limiting examples of suitable inducible promoters include those from genes such as cytochrome P450 genes, heat shock protein genes, metallothionein genes, and hormone-inducible genes, such as the estrogen gene promoter. Another example of an inducible promoter is the tetVP16 promoter that is responsive to tetracycline.

Several promoters are publicly available or described. For example, Ple155 promoter is available through Addgene plasmid repository (Addgene plasmid #29011) and is described in Scalabrino, et al. (Hum Mol Genet. (2015), 24 (21): 6229-39). Ye, et al. (Hum Gene Ther. (2016); 27(1):72-82) describes a shorter version of this promoter called PR1.7. A Thy l promoter construct is also available through Addgene plasmid repository (Addgene plasmid #20736). A GRM6 promoter construct is also available through Addgene plasmid repository (Addgene plasmid #66391). Guziewicz, et al. (PLOS One (2013); 8 (10): e75666) and Esumi, et al. (J Biol Chem. (2004), 279 (18): 19064-73) provide examples of the use of VMD2 promoter. Dyka, et al. (Adv Exp Med Biol. (2014); 801:695-701) describes cone-specific promoters for use in gene therapy, including IRBP and IRBPe-GNAT2 promoter. The use of PR2.1 promoter has been demonstrated in Komáromy, et al. (Gene Ther.(2008); 15 (14): 1049-55) and its characterization in Karim, et al. (Tree Physiol.(2015); 35 (10): 1129-39). Aartsen, et al. (PLOS One, (2010); 5(8):e12387) describes the use of GFAP promoter to drive GFP expression in Müller glial cells. Other examples of Müller glia specific promoters are RLBP1 and GLAST (Vázquez-Chona, Invest Ophthalmol Vis Sci. (2009), 50(8):3996-4003; Regan, et al., J Neurosci, (2007), 27(25): 6607-6619).

Synthetic promoters are also contemplated herein. A synthetic promoter may comprise, for example, regions of known promoters, regulatory elements, transcription factor binding sites, enhancer elements, repressor elements, and the like.

It is to be understood that a promoter may be a fragment of any one of the promoters disclosed herein, or one that retains partial promoter activity (e.g., 10-90, 30-60, 50-80, 80-99 or 90-99.9% of the activity) of a whole promoter.

In some embodiments, an AAV genome is built of single-stranded deoxyribonucleic acid (ssDNA), which is either positive- or negative-sensed. At each end of the DNA strand is an inverted terminal repeat (ITR). Between the ITRs are two open reading frames (ORFs): rep and cap. The rep ORF is composed of four overlapping genes encoding Rep proteins required for the AAV life cycle. The cap ORF contains overlapping nucleotide sequences of capsid proteins: VP1, VP2 and VP3, which interact together to form a capsid of an icosahedral symmetry.

In some embodiments, the nucleic acid encoding the gene of interest (e.g., along with a promoter) can be inserted in between two ITRs (e.g., by replacing one or more of the viral genes). Accordingly, in some embodiments, a gene of interest is flanked by inverted terminal repeats (ITRs), e.g., AAV ITRs, ITRs from AAV9 or any other AAV serotype including but not limited to AAV1, AAV2, AAV2.5, AAV3, AAV4, AAV5, AAV6, AAV7, AAV8, AAVrh.10, AAVrh.74, AAV10, AAV11, AAV12, AAV-DJ, PHP.eB, AvianAAV, or AAV13. In some embodiments, a recombinant rAAV particle comprises a nucleic acid vector, such as a single-stranded (ss) or self-complementary (sc) AAV nucleic acid vector. In some embodiments, the nucleic acid vector contains an expression construct as described herein and one or more regions comprising inverted terminal repeat (ITR) sequences (e.g., wild-type ITR sequences or engineered ITR sequences) flanking the expression construct. In some embodiments, the nucleic acid is encapsidated by a viral capsid.

### Variants

Aspects of the disclosure relate to recombinant AAV9 capsid proteins. Variants of the capsid proteins described in this application are also encompassed by this disclosure. A variant may share at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with a reference sequence, including all values in between. In some embodiments, the disclosure provides variants of an AAV9 capsid protein.

Unless otherwise noted, the term “sequence identity,” as known in the art, refers to a relationship between the sequences of two polypeptides (or polynucleotides), as determined by sequence comparison (alignment). In some embodiments, sequence identity is determined across the entire length of a sequence, while in other embodiments, sequence identity is determined over a region of a sequence. “Identity” can also refer to the degree of sequence relatedness between two sequences as determined by the number of matches between strings of two or more residues (e.g., amino acid residues). Identity measures the percent of identical matches between the smaller of two or more sequences with gap alignments (if any) addressed by a particular mathematical model, algorithms, or computer program.

It will be appreciated that when a sequence of a first, shorter length is aligned with a sequence of a second, longer length, the resultant alignment may contain gaps in the first sequence that account for the relative difference in length between the two sequences. An “aligned sequence” or the “alignment of” a sequence, relative to another sequence, may include gaps or spaces, as necessary for the alignment of interest.

The identity of related polypeptide sequences can be readily calculated by any of the methods known to one of ordinary skill in the art. In some embodiments, the “percent identity” of two sequences (e.g., amino acid sequences) is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul et al., J. Mol. Biol. 215:403-10, 1990. Where gaps exist between two sequences, Gapped BLAST can be utilized, for example, as described in Altschul et al., Nucleic Acids Res. 25(17):3389-3402, 1997. When utilizing BLAST and Gapped BLAST programs, the default parameters of the respective programs (e.g., XBLAST and NBLAST) can be used, or the parameters can be adjusted appropriately as would be understood by one of ordinary skill in the art.

Another local alignment technique which may be used, for example, is based on the Smith-Waterman algorithm (Smith, T.F. & Waterman, M.S. (1981) “Identification of common molecular subsequences.” J. Mol. Biol. 147:195-197). A general global alignment technique which may be used, for example, is the Needleman-Wunsch algorithm (Needleman, S.B. & Wunsch, C.D. (1970) “A general method applicable to the search for similarities in the amino acid sequences of two proteins.” J. Mol. Biol. 48:443-453), which is based on dynamic programming.

More recently, a Fast Optimal Global Sequence Alignment Algorithm (FOGSAA) was developed that purportedly produces global alignment of nucleic acid and amino acid sequences faster than other optimal global alignment methods, including the Needleman-Wunsch algorithm. In some embodiments, the identity of two polypeptides is determined by aligning the two amino acid sequences, calculating the number of identical amino acids, and dividing by the length of one of the amino acid sequences.

For multiple sequence alignments, computer programs including Clustal Omega (Sievers et al., Mol Syst Biol. 2011 Oct. 11; 7:539) may be used. In some embodiments, an amino acid sequence is found to have a specified percent identity to a reference sequence, such as a sequence disclosed in this application and/or recited in the claims, when sequence identity is determined using Clustal Omega (Sievers et al., Mol Syst Biol. 2011 Oct. 11; 7:539).

### Recombinant AAV Particle Compositions

Provided herein is a composition comprising any one of the recombinant AAV9 particles disclosed herein.

In some embodiments, any one of the compositions provided herein comprises a pharmaceutically acceptable carrier. The term “carrier” refers to a diluent, adjuvant, excipient, or vehicle with which the rAAV particle is administered to a subject. Such pharmaceutical carriers can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions can also be employed as liquid carriers. Non-limiting examples of pharmaceutically acceptable carriers include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, polyacrylic acids, lubricating agents (such as talc, magnesium stearate, and mineral oil), wetting agents, emulsifying agents, suspending agents, preserving agents (such as methyl-, ethyl-, and propyl-hydroxy-benzoates), and pH adjusting agents (such as inorganic and organic acids and bases). Other examples of carriers include phosphate buffered saline, HEPES-buffered saline, and water for injection, any of which may be optionally combined with one or more of calcium chloride dihydrate, disodium phosphate anhydrous, magnesium chloride hexahydrate, potassium chloride, potassium dihydrogen phosphate, sodium chloride, or sucrose. Other examples of carriers that might be used include saline (e.g., sterilized, pyrogen-free saline), saline buffers (e.g., citrate buffer, phosphate buffer, acetate buffer, and bicarbonate buffer), amino acids, urea, alcohols, ascorbic acid, phospholipids, proteins (for example, serum albumin), EDTA, sodium chloride, liposomes, mannitol, sorbitol, and glycerol. USP grade carriers and excipients are particularly useful for delivery of rAAV particles to human subjects.

Typically, such compositions may contain at least about 0.1% of the therapeutic agent (e.g., rAAV particle) or more, although the percentage of the active ingredient(s) may, of course, be varied and may conveniently be between about 1 or 2% and about 70% or 80% or more of the weight or volume of the total formulation. Naturally, the amount of therapeutic agent(s)(e.g., rAAV particle) in each therapeutically-useful composition may be prepared in such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be designed.

### Methods of Delivering a Protein of Interest to a Subject

Subjects previously treated with AAV particles develop neutralizing antibodies against those particles. In such instances, treating the subject again with AAV particles that would react or even cross-react with the developed neutralizing antibodies results in an unwanted antigenic response. Any one of the compositions comprising any one of the variant AAV9 particles that can escape neutralizing antibodies, as described herein, would be useful as a vehicle for gene delivery in such instances. Accordingly, provided herein is a method of delivering a protein of interest to a subject, the method comprising administering to the subject a composition comprising any one of the AAV9 particles disclosed herein. Such a method diminishes the immunogenic response to the administered AAV9 particles used to deliver a therapeutic gene.

Provided herein is also a method of reducing the antigenic response to AAV9 particles administered to a subject. In some embodiments, the antigenic response to any one of the variant AAV9 particles disclosed herein decreases by 5-100% (e.g., 5-100, 5-10, 10-30, 20-50, 20-70, 50-100, 5-60, 20-80 or 80-100%) relative to the antigenic response to a wild-type AAV9 particle in the same or same type of subject. A same type of subject may have the same neutralizing antibodies.

As used herein, “administering” or “administration” means providing a material to a subject in a manner that is pharmacologically useful. In some embodiments, a subject is a mammal. In some embodiments, a mammalian subject is a human, a non-human primate, or a non-primate mammal, such as a dog, a cat, a hamster, a mouse, a rat, a pig, a horse, a cow, a donkey or a rabbit. In some embodiments, the subject is a human.

In certain circumstances it will be desirable to deliver the rAAV particles in suitably formulated pharmaceutical compositions disclosed herein either subcutaneously, intraocularly, intravitreally, subretinally, parenterally, intravenously (IV), intracerebro-ventricularly, intramuscularly, intrathecally (IT), intracisternally, orally, intraperitoneally, by oral or nasal inhalation, or by direct injection to one or more cells, tissues, or organs by direct injection. In some embodiments, the administration is a route suitable for systemic delivery, such as by intravenous injection.

In some embodiments, a rAAV9 particle as described herein is administered to a subject to treat a disease or disorder. To “treat” a disease or disorder, as the term is used herein, means to reduce the frequency or severity of at least one sign or symptom of a disease or disorder experienced by a subject. The compositions described above or elsewhere herein are typically administered to a subject in an effective amount, that is, an amount capable of producing a desirable result. The desirable result will depend upon the active agent being administered. For example, an effective amount of rAAV9 particles may be an amount of the particles that is capable of transferring an expression construct to a host organ, tissue, or cell. A therapeutically acceptable amount may be an amount that is capable of treating a disease, e.g., a spinal muscular atrophy (SMA). As is well known in the medical and veterinary arts, dosage for any one subject depends on many factors, including the subject's size, body surface area, age, the particular composition to be administered, the active ingredient(s) in the composition, time and route of administration, general health, and other drugs being administered concurrently.

In some embodiments, the concentration of rAAV particles administered to a subject may be on the order ranging from 10⁶ to 10¹⁴ particles/ml or 10³ to 10¹⁵ particles/ml, or any values therebetween for either range, such as for example, about 10³, 10⁴, 10⁵, 10⁶, 10⁷, 10⁸, 10⁹, 10¹⁰, 10¹¹, 10¹², 10¹³, 10¹⁴, or 10¹⁵ particles/ml. In some embodiments, rAAV particles of a higher concentration than 10¹³ particles/ml are administered. In some embodiments, rAAV particles of a higher concentration than 10¹⁵ particles/ml are administered. In some embodiments, the concentration of rAAV particles administered to a subject may be on the order ranging from 10⁶ to 10¹⁴ vector genomes (vgs)/ml or 10³ to 10¹⁵ vgs/ml, or any values therebetween for either range (e.g., 10³, 10⁴, 10⁵, 10⁶, 10⁷, 10⁸, 10⁹, 10¹⁰, 10¹¹, 10¹², 10¹³, 10¹⁴, or 10¹⁵ vgs/ml). In some embodiments, rAAV particles of higher concentration than 10¹³ vgs/ml are administered. In some embodiments, rAAV particles of a higher concentration than 10¹⁵ particles/ml are administered. The rAAV particles can be administered as a single dose, or divided into two or more administrations as may be required to achieve therapy of the particular disease or disorder being treated. In some embodiments, 0.0001 ml to 10 ml are delivered to a subject. In some embodiments, the number of rAAV particles administered to a subject may be on the order ranging from 10⁶-10¹⁴ vg/kg, or any values therebetween (e.g., 10⁶, 10⁷, 10⁸, 10⁹, 10¹⁰, 10¹¹, 10¹², 10¹³, or 10¹⁴ vgs/mg). In some embodiments, the dose of rAAV particles administered to a subject may be on the order ranging from 1012-10¹⁴ vgs/kg. In some embodiments, the volume of rAAV9 composition delivered to a subject (e.g., via one or more routes of administration as described herein) is 0.0001 ml to 10 ml.

In some embodiments, a composition disclosed herein (e.g., comprising a rAAV9 particle) is administered to a subject once. In some embodiments, the composition is administered to a subject multiple times (e.g., twice, three times, four times, five times, six times, or more). Repeated administration to a subject may be conducted at a regular interval (e.g., daily, every other day, twice per week, weekly, twice per month, monthly, every six months, once per year, or less or more frequently) as necessary to treat (e.g., improve or alleviate) one or more symptoms of a disease, disorder, or condition in the subject. In some embodiments, administration of a rAAV9 particle to a subject results in the prevention, alleviation or amelioration of one or more signs or symptoms of a disease or disorder.

In some embodiments, the subject has or is suspected of having a disease or disorder that may be treated with gene therapy. Non-limiting examples of conditions for which rAAV-based gene therapy may find particular utility include, but are not limited to, cancer, diabetes, autoimmune disease, neurological disease, brain disease, spinal disease, kidney disease, cardiovascular disease, pancreatic disease, intestinal disease, liver disease, hemophilia, α1-antitrypsin (AAT) deficiency, ischemia, skeletal disease and pulmonary disease. In some embodiments, the disease or disorder is selected from the group consisting of: Batten Disease, Canavan Disease, Pompe Disease, GM1 Gangliosidosis, Wilson Disease, Glycogen Storage Disease Type Ia, Ornithine Transcarbamylase Deficiency, liver fibrosis, Hemophilia A, Hemophilia B, and Hemophilia C. In some embodiments, the disease or disorder is a cancer. In some embodiments, the cancer is one or more of cancer of the buccal cavity or pharynx, cancer of the digestive tract, cancer of the colon, rectum, or anus, cancer of the respiratory tract, breast cancer, cancer of the cervix, uteri, vagina, or vulva, cancer of the uterine corpus or ovary, cancer of the male genital tract, cancer of the urinary tract, bone or soft tissue cancer, Kaposi sarcoma, melanoma of the skin, ocular melanoma, non-melanoma eye cancer, cancer of the brain and central nervous system, cancer of the thyroid and other endocrine glands, Hodgkin Lymphoma, Non-Hodgkin Lymphoma, myeloma, renal cancer, colorectal cancer, lung cancer, breast cancer, pancreatic cancer, prostate cancer, gastric cancer, GIST or glioblastoma.

In some embodiments, the subject has or is suspected of having spinal muscular atrophy (SMA).

In some embodiments, the method comprises contacting a cell with a rAAV9 particle as described herein. In some embodiments, a cell disclosed herein is a cell isolated or derived from a subject. In some embodiments, a cell is a mammalian cell (e.g., a cell isolated or derived from a mammal). In some embodiments, the cell is a human cell, non-human primate cell, rat cell, or mouse cell. In some embodiments, a cell is isolated or derived from a particular tissue of a subject, such as liver tissue. In some embodiments, the cell is a liver, brain, heart or retina cell. In some embodiments, a cell is a liver cell. In some embodiments, a cell is in vitro. In some embodiments, a cell is ex vivo. In some embodiments, a cell is in vivo. In some embodiments, a cell is within a subject (e.g., within a tissue or organ of a subject). In some embodiments, a cell is a primary cell. In some embodiments, a cell is from a cell line (e.g., an immortalized cell line). In some embodiments a cell is a cancer cell or an immortalized cell.

Methods of contacting a cell may comprise, for example, contacting a cell in a culture with a rAAV9 particle as described herein. In some embodiments, contacting a cell comprises adding a rAAV9 particle as described herein to the supernatant of a cell culture (e.g., a cell culture on a tissue culture plate or dish) or mixing a rAAV9 particle as described herein with a cell culture (e.g., a suspension cell culture). In some embodiments, contacting a cell comprises mixing a rAAV9 particle as described herein with another solution, such as a cell culture media, and incubating a cell with the mixture.

In some embodiments, contacting a cell with a rAAV9 particle as described herein comprises administering a rAAV9 particle as described herein to a subject or device in which the cell is located. In some embodiments, contacting a cell comprises injecting a rAAV9 particle as described herein into a subject in which the cell is located. In some embodiments, contacting a cell comprises administering a rAAV9 particle as described herein directly to a cell, or into or substantially adjacent to a tissue of a subject in which the cell is present.

Vectors and Kits Useful in the Preparation of rAAV Particles

In some embodiments, provided herein is a vector comprising a nucleic acid encoding one or more AAV9 Cap proteins with any one or more of the mutations described herein. Such a vector can be used to prepare or package any one of the AAV9 particles disclosed herein. In some embodiments, provided herein is a vector comprising a nucleic acid encoding one or more genes of interest and/or one or more promoters. In some embodiments, a vector is a plasmid. In some embodiments, a vector (e.g., a plasmid) is provided in a host cell.

Methods of producing rAAV particles are known in the art (see, e.g., Zolotukhin, et al., Methods (2002); 28 (2): 158-67; and U.S. Patent Publication Numbers US20070015238 and US20120322861, which are incorporated herein by reference; and plasmids and kits available from ATCC and Cell Biolabs, Inc.). For example, a plasmid comprising a gene of interest may be combined with one or more helper plasmids, e.g., that contain a rep gene (e.g., encoding Rep78, Rep68, Rep52 and Rep40) and transfected into recombinant cells such that the recombinant AAV particle can be packaged and subsequently purified.

In some embodiments, the one or more helper plasmids include a first helper plasmid comprising a rep gene, a nucleic acid encoding any one of the Cap proteins disclosed herein, and a second helper plasmid comprising one or more of the following helper genes: E1a gene, E1b gene, E4 gene, E2a gene, and VA gene. For clarity, helper genes are genes that encode helper proteins E1a, E1b, E4, E2a, and VA.

In some embodiments, the packaging is performed in a helper cell or producer cell, such as a mammalian cell or an insect cell. Non-limiting examples of mammalian cells are HEK293 cells, COS cells, HeLa cells, BHK cells, or CHO cells (see, e.g., ATCC® CRL-1573™, ATCC® CRL-1651™, ATCC® CRL-1650™, ATCC® CCL-2, ATCC® CCL-10™, or ATCC® CCL-61™). A non-limiting example of an insect cell is Sf9 cells (see, e.g., ATCC® CRL-1711™). A helper cell may comprise rep genes that encode the Rep proteins for use in a method described herein. In some embodiments, the packaging is performed in vitro.

Provided herein is also a kit that comprises tools for the preparing of any one of the AAV9 particles provided herein. Such a kit comprises any one of the vectors encoding any one of the AAV9 Cap proteins described herein, as a first component. A kit may also comprise a vector comprising AAV helper genes as a second component, wherein each component is packaged in separate containers. In some embodiments, a kit also comprises AAV packaging cells that are provided in a third container that is separate from the first and second containers.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

The present invention is further illustrated by the following Examples, which should not be construed as limiting. The entire contents of all of the references (including literature references, issued patents, published patent applications, and co-pending patent applications) cited throughout this application are hereby expressly incorporated by reference. If a reference incorporated in this application contains a term whose definition is incongruous or incompatible with the definition of same term as defined in the present disclosure, the meaning ascribed to the term in this disclosure shall govern. Mention of any reference, article, publication, patent, patent publication, and patent application cited in this application is not, and should not be taken as, an acknowledgment or suggestion that they constitute valid prior art or form part of the common general knowledge of a skilled artisan.

### Examples

In order that the invention described in this application may be more fully understood, the following examples are set forth. The examples described in this application are offered to illustrate the systems and methods provided in this application and are not to be construed as limiting their scope.

### Example 1: Use of Patient-Derived mAbs to Design AAV9 Capsid Variants Capable of Escaping Neutralizing Antibodies

The use of vectors based on adeno-associated virus 9 (AAV9) for gene therapy applications has grown rapidly over recent years, especially following the FDA approval of an AAV9-based biologic for the treatment of spinal muscular atrophy. In addition, there is interest in broader AAV9 usage due to its ability to transduce cardiac and skeletal muscle, liver, pancreas, the eye and its capability to cross the blood-brain barrier to transduce the central nervous system (CNS). However, the presence of pre-existing neutralizing antibodies (Nabs) against AAV9 capsids in a large percentage of the population excludes many patients from being treated. A strategy to circumvent the immune response of a patient for AAV-mediated therapeutic gene delivery is the development of modified vector capsids, able to escape antibody recognition, by either directed evolution or rational capsid design. However, in order to pursue this strategy, the antigenic regions of the capsids need to be characterized and the contact residues identified to which the Nabs bind. Previous work generated mouse monoclonal antibodies targeting the AAV capsids to simulate the immune response against the capsid and to map their major antigenic regions. However, mouse antibodies may not fully mimic human-derived antibodies.

To address the potential issues with mouse antibodies, human monoclonal antibodies were structurally characterized that were obtained from patients that received an AAV9-based biologic for the treatment of spinal muscular atrophy. The antibody binding sites on the AAV9 capsid were determined by cryo-electron microscopy (cryo-EM) and three-dimensional image reconstruction of 21 antibodies obtained from three patients, who were enrolled into a clinical trial for an AAV9-based biologic for the treatment of spinal muscular atrophy. All the antibodies neutralized AAV9 transduction, and some also cross-reacted and neutralized a range of different AAV serotypes. The structural information of these capsid-antibody complexes was used to generate amino acid substitutions on the capsid surface of AAV9. The new variants were initially tested for their productivity and infectivity, and subsequently for their ability to escape the neutralizing antibodies. Results of these initial tests, as well as an exemplary list of mutations, are shown in FIGS. 3A and 3B. Individual amino acid substitutions were combined to generate AAV9 capsid variants capable of escaping multiple, most, or all neutralizing antibodies.

These engineered AAV9 capsids expand the pool of potential patients eligible for AAV gene delivery. Moreover, the new capsid variants may allow redosing of patients previously treated with AAV9 vectors.

The new capsids are the first engineered AAV variants based on neutralizing, human antibodies.

Production of rAAV

rAAV production may be assessed by conventional means. rAAV may be produced in human cells (for example, HEK293 cells) that are transiently transfected with plasmids. rAAV may also be produced in insect cells using baculoviruses. Recombinant HSV complementation systems in suspension-cultured mammalian cells (such as BHK21 or HEK23 cells) and mammalian-derived producer cell lines containing rep and cap genes and the AAV vector integrated into the genome may also be used.

Production efficiency may be assessed by comparing the yield produced by a variant of interest relative to wild-type AAV9 capsids. This can be quantified by quantitative PCR (qPCR) detecting the packaged transgene.

### Neutralization Assays

Neutralization of viral particles by antibodies may be assessed by conventional means. Briefly, a virus of interest is incubated with neutralizing antibody and added to susceptible cells. The infectivity of the virus is then assessed following a 2-day incubation period using a reporter gene assay (for example, an assay measuring GFP fluorescence or luciferase light generation). The results are compared to virus not exposed to the neutralizing antibody or to other capsid variants capable of escaping the neutralizing antibodies.

### Example 2: Anti-AAV Capsid Monoclonal Antibodies Show Variable Cross-Reactivity

Seven mAbs were selected per patient, and purified mAb stocks were prepared for characterization. The mAbs were screened by native dot immunoblot to explore cross-reactivity to other natural AAV serotypes.

### Methods

AAV capsids were adsorbed onto nitrocellulose membranes (Bio-Rad, Hercules, CA) in a dot blot manifold (Schleicher and Schuell, Dassel, Germany). Excess fluid was drawn through the membrane by vacuum filtration. The membrane was removed from the manifold and blocked with 6% milk in PBS, pH 7.4 for 1 h. Primary antibody in the form of hybridoma supernatant was applied to the membrane at a 1:50 dilution in PBS with 6% milk, 0.1% Tween-20 and incubated for 1 hr. The membrane was then washed with PBS and horse radish peroxidase (HRP)-linked secondary antibody (GE Healthcare) was applied at a dilution of 1:3000 in PBS and incubated for 1 h. The membrane was washed with PBS and then Immobilon™ Chemiluminescent Substrate (Millipore, Darmstadt, Germany) was applied to the membrane and the signal detected on X-ray film.

### Results

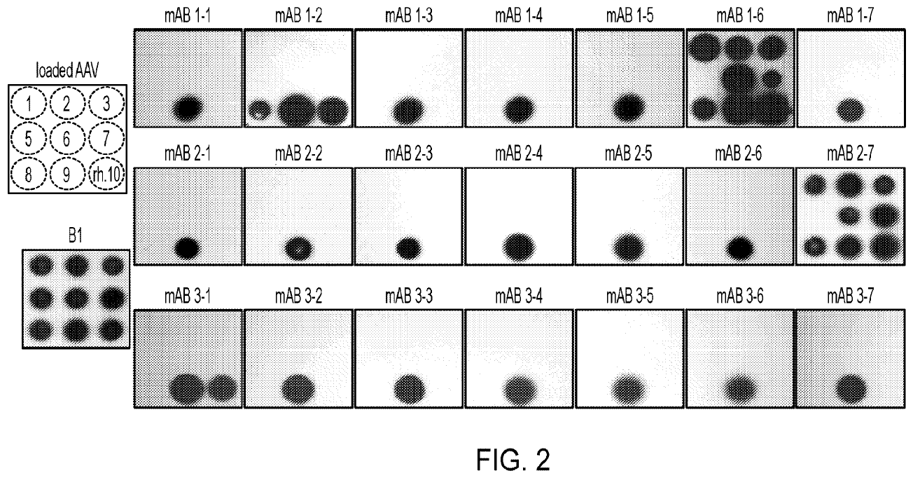

The assay was performed against serotypes 1, 2, 3, 5, 6, 7, 8, 9 and rh10 with the majority of mAbs (17 of 21) being AAV9 specific (FIG. 2). Four mAbs showed cross-reactivity to other capsid serotypes with mAb1-2 cross-reacting with AAV8 and AAVrh10, mAb3-1 cross-reacting with AAVrh10 and two mAbs, mAb1-6 and mAb2-7, cross-reacting with all serotypes except the phylogenetically distant AAV5 capsid.

### Example 3: Cryo-EM Mapping

Fabs were prepared for the 21 mAbs from Example 2 and mixed with AAV9 to map their binding sites to the surface of the capsids. Cryo-EM data was collected of the complexed samples and 3D image reconstruction utilized to generate ~5-10 Å resolution complex maps from ~2,000 to 10,000 individual particles.

### Methods

Purified AAV9 capsids were mixed with Fabs at a molecular ratio of ~2 Fabs per potential VP binding site in the 60-meric capsid, giving a final ratio of ~1:120 (capsid to Fab). Aliquots (3.5 μl) of the purified samples either with or without Fabs were applied to glow-discharged Quantifoil copper grids with 2 nm continuous carbon support over holes (Quantifoil R 2/2 400 mesh), blotted, and vitrified using a Vitrobot Mark 4 (FEI) at 95% humidity and 4° C. The grids were imaged using a FEI Tecnai G2 F20-TWIN microscope (FEI) operated under low-dose conditions (200 kV,~20e-/Å2). Images were collected on a GatanUltraScan 4000 CCD camera (Gatan) at a pixel size of 1.82 Å. Data for the samples utilizing the Fabs from patient 3 (Fab3-1 to Fab3-7) and Fab1-7 were collected at the Stanford-SLAC Cryo-EM Center (S2C2) using a Titan Krios (FEI) electron microscope operated at 300 kV equipped with a Falcon 4 direct electron detector (Thermo Fisher). 50 movie frames were collected per micrograph at a total electron dose of ~50 e-/Å2 and a pixel size of 0.95 Å. Motion corrected micrographs were obtained by aligning the movie frames using MotionCor2 with dose weighting. In order to obtain ~5-6 Å structures the micrographs were binned utilizing a bin-factor of 3. For 3D image reconstruction the cisTEM software package was utilized and the data processed a described previously. The resolution of the cryo-reconstructed density maps was estimated based on a Fourier Shell Correlation of 0.143. The density maps were analyzed using Chimera.

### Results

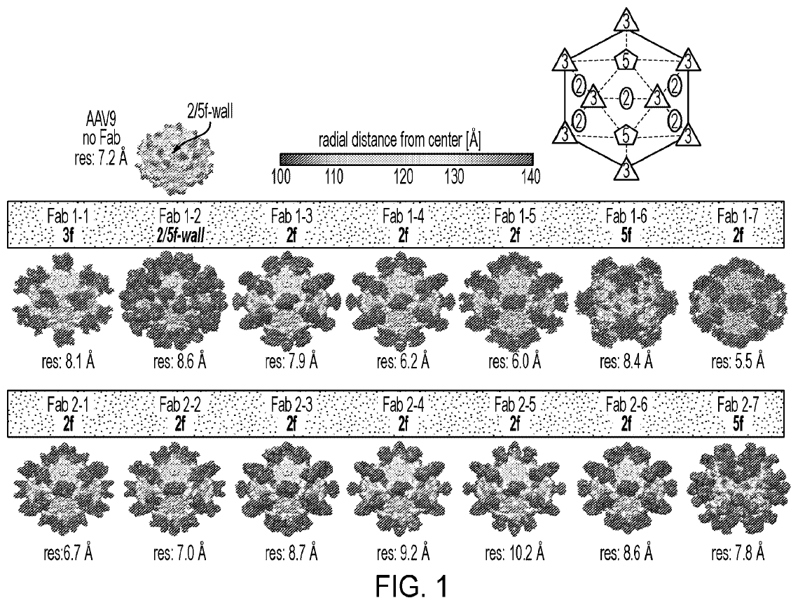

Fabs were found to bind either the 2-, 3-, 5-fold axis of symmetry or the 2/5-fold wall of the AAV9 capsid (FIG. 1). However, while the Fabs showed binding to all four topological regions, Fabs binding near or across the 2-fold symmetry axis were the most prevalent, as ~76% (n=16) of Fabs bound to this region. This dominant binding pattern was observed for all three patients (Pt1:57%, Pt2:86%, Pt3:86%). Among the 2f-binding Fabs different interacting conformations were observed (for example: Fab1-3 vs. Fab1-7 vs. Fab2-1). For the non-2f-binding Fabs, two were shown to bind to the center of the 3-fold axis of symmetry (Fab1-1, Fab3-4), one to the 2/5-fold wall (Fab1-2), and two to the 5-fold region (Fab1-6 and Fab2-7) with different binding conformations. In summary, the Fabs derived from the 21 human mAbs bound either of the four topological regions of the capsid surface but showed antigenic dominance to the 2-fold region of the AAV9 capsid.

### Example 4: Cryo-Reconstructed AAV9-Fab Complexes

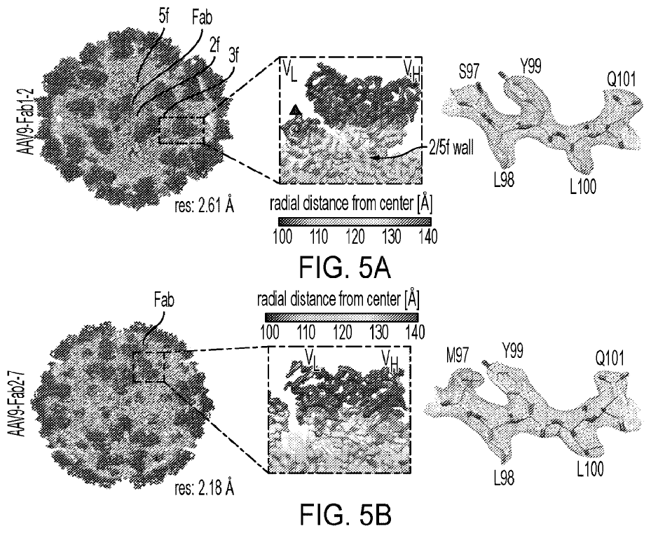

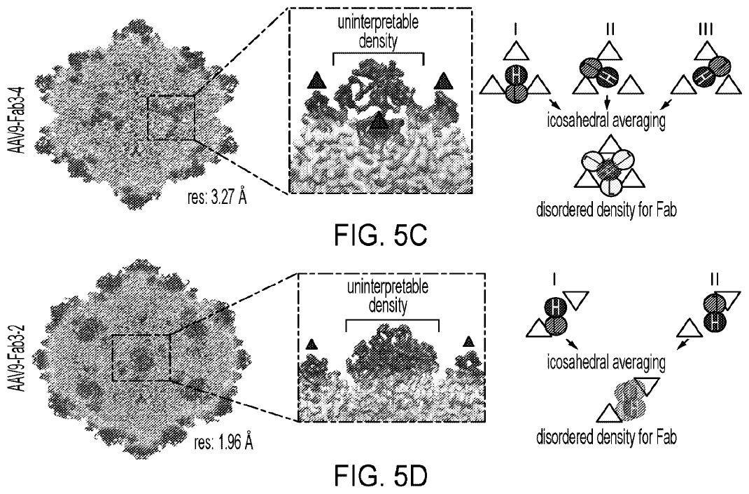

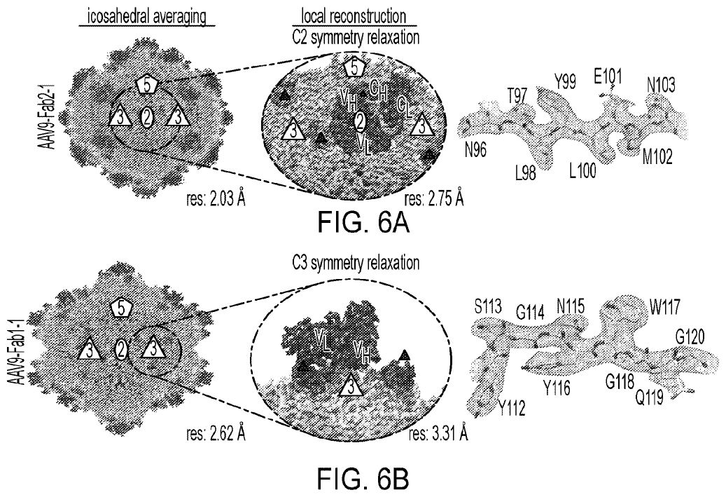

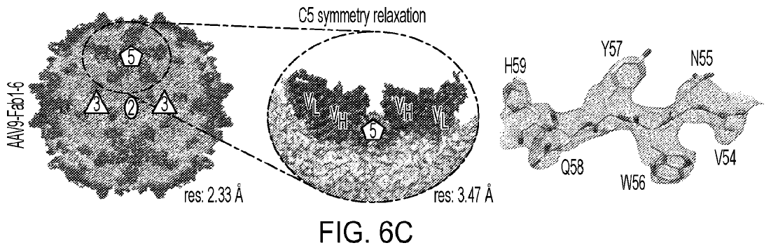

Cryo-EM was used as described above to further characterize the interaction between the AAV9 capsid and Fabs of interest. Results are shown in FIGS. 5A-5D and FIGS. 6A-6C. A summary of the data collection, image processing and refinement statistics is show in the table below.

| | 1-1 | 1-2 | 1-3 | 1-4 | 1-5 | 1-6 | 1-7 |
|---|---|---|---|---|---|---|---|
| Total number of | 4421 | 3790 | 7579 | 8998 | 5553 | 5831 | 5350 |
| micrographs | | | | | | | |
| Pixel size | 0.83 | 0.83 | 0.84 | 0.83 | 0.83 | 0.83 | 0.72 |
| (Å/pixel) | | | | | | | |
| Capsid # icos. | 95,487 | 173,130 | 157,695 | 150,950 | 155,810 | 245,942 | 223,591 |
| map | | | | | | | |
| Resolution icos. | 2.62 | 2.61 | 1.99 | 2.46 | 2.58 | 2.33 | 1.88 |
| map (Å) | | | | | | | |
| Subparticles # | 691,908 | N/A | 410,892 | 1,141,027 | 283,502 | 89,904 | 428,054 |
| local map | | | | | | | |
| Resolution local | 3.31 | N/A | 3.13 | 2.77 | 3.19 | 3.47 | 2.54 |
| map (Å) | | | | | | | |
| refinement | | | | | | | |
| statistics | | | | | | | |
| Map CC | 0.858 | 0.892 | 0.730 | 0.836 | 0.832 | 0.816 | 0.853 |
| RMSD bonds | 0.01 | 0.01 | 0.01 | 0.01 | 0.01 | 0.01 | 0.01 |
| (Å) | | | | | | | |
| RMSD angles | 0.80 | 0.85 | 0.75 | 0.79 | 0.73 | 1.01 | 0.80 |
| (°) | | | | | | | |
| All-atom | 9.22 | 7.57 | 13.64 | 8.04 | 9.80 | 13.99 | 10.44 |
| clashscore | | | | | | | |
| Ramachandran | | | | | | | |
| plot | | | | | | | |
| Outliers | 0 | 0 | 0.1 | 0 | 0 | 0.3 | 0 |
| Allowed | 3.2 | 3.5 | 5.3 | 3.5 | 4.6 | 4.7 | 4.3 |
| Favored | 96.8 | 96.5 | 94.6 | 96.5 | 95.4 | 95.0 | 95.7 |
| Rotamer outliers | 0 | 0 | 0 | 0 | 0 | 0 | 0 |
| C-beta | 0 | 0 | 0 | 0 | 0 | 0 | 0 |
| deviations | | | | | | | |

| | 2-1 | 2-2 | 2-3 | 2-4 | 2-5 | 2-6 | 2-7 |
|---|---|---|---|---|---|---|---|
| Total number of | 8791 | 7630 | 7170 | 7787 | 4767 | 2859 | 6232 |
| micrographs | | | | | | | |
| Pixel size | 0.93 | 0.83 | 0.83 | 0.83 | 0.83 | 0.83 | 0.83 |
| (Å/pixel) | | | | | | | |
| Capsid # icos. | 366,468 | 143,856 | 148,486 | 131,839 | 77,531 | 239,814 | 263,350 |
| map | | | | | | | |
| Resolution icos. | 2.03 | 2.03 | 2.03 | 2.18 | 2.68 | 2.24 | 2.18 |
| map (Å) | | | | | | | |
| Subparticles # | 541,473 | 1,085,751 | 1,164,081 | 360,595 | 499,674 | 2,092,947 | N/A |
| local map | | | | | | | |
| Resolution local | 2.75 | 2.82 | 2.82 | 3.02 | 2.86 | 2.98 | N/A |
| map (Å) | | | | | | | |
| refinement | | | | | | | |
| statistics | | | | | | | |
| Map CC | 0.834 | 0.834 | 0.848 | 0.807 | 0.832 | 0.775 | 0.837 |
| RMSD bonds | 0.01 | 0.01 | 0.00 | 0.01 | 0.01 | 0.02 | 0.01 |
| (Å) | | | | | | | |
| RMSD angles | 0.86 | 0.86 | 0.69 | 0.74 | 0.70 | 0.85 | 0.96 |
| (°) | | | | | | | |
| All-atom | 8.87 | 8.94 | 8.96 | 9.94 | 9.62 | 12.33 | 6.22 |
| clashscore | | | | | | | |
| Ramachandran | | | | | | | |
| plot | | | | | | | |
| Outliers | 0 | 0 | 0 | 0 | 0 | 0 | 0 |
| Allowed | 3.4 | 3.6 | 4.6 | 4.1 | 3.4 | 4.2 | 3.7 |
| Favored | 96.6 | 96.4 | 95.4 | 95.9 | 96.6 | 95.8 | 96.3 |
| Rotamer outliers | 0 | 0 | 0 | 0 | 0 | 0 | 0.2 |
| C-beta | 0 | 0 | 0 | 0 | 0 | 0 | 0 |
| deviations | | | | | | | |

| | 3-1 | 3-2 | 3-3 | 3-4 | 3-5 | 3-6 | 3-7 |
|---|---|---|---|---|---|---|---|
| Total number of | 5320 | 4731 | 4604 | 3180 | 3831 | 3226 | 3799 |
| micrographs | | | | | | | |
| Pixel size | 0.72 | 0.72 | 0.93 | 0.93 | 0.93 | 0.93 | 0.93 |
| (Å/pixel) | | | | | | | |
| Capsid # icos. | 79,023 | 52,299 | 70,076 | 112,084 | 155,703 | 43,865 | 109,464 |
| map | | | | | | | |
| Resolution icos. | 2.84 | 1.96 | 2.39 | 3.27 | 2.02 | 2.02 | 2.41 |
| map (Å) | | | | | | | |
| Subparticles # | 220,250 | 79,771 | 185,536 | 622,842 | 601,051 | 480,867 | 1,008,032 |
| local map | | | | | | | |
| Resolution local | 3.02 | 2.84 | 3.56 | 3.73 | 2.76 | 3.36 | 2.76 |
| map (Å) | | | | | | | |
| refinement | | | | | | | |
| statistics | | | | | | | |
| Map CC | 0.809 | 0.839 | 0.822 | 0.846 | 0.849 | 0.766 | 0.791 |
| RMSD bonds | 0.01 | 0.01 | 0.01 | 0.01 | 0.01 | 0.01 | 0.01 |
| (Å) | | | | | | | |
| RMSD angles | 0.80 | 0.83 | 0.81 | 0.68 | 0.81 | 0.89 | 0.80 |
| (°) | | | | | | | |
| All-atom | 9.96 | 9.54 | 11.03 | 9.68 | 6.96 | 11.88 | 10.11 |
| clashscore | | | | | | | |
| Ramachandran | | | | | | | |
| plot | | | | | | | |
| Outliers | 0.1 | 0 | 0 | 0.1 | 0 | 0.1 | 0 |
| Allowed | 4.5 | 4.0 | 5.1 | 4.0 | 3.5 | 5.1 | 3.3 |
| Favored | 95.4 | 96.0 | 94.9 | 95.9 | 96.5 | 94.8 | 96.7 |
| Rotamer outliers | 0 | 0 | 0 | 0 | 0 | 0 | 0.0 |
| C-beta | 0 | 0 | 0 | 0 | 0 | 0 | 0 |
| deviations | | | | | | | |

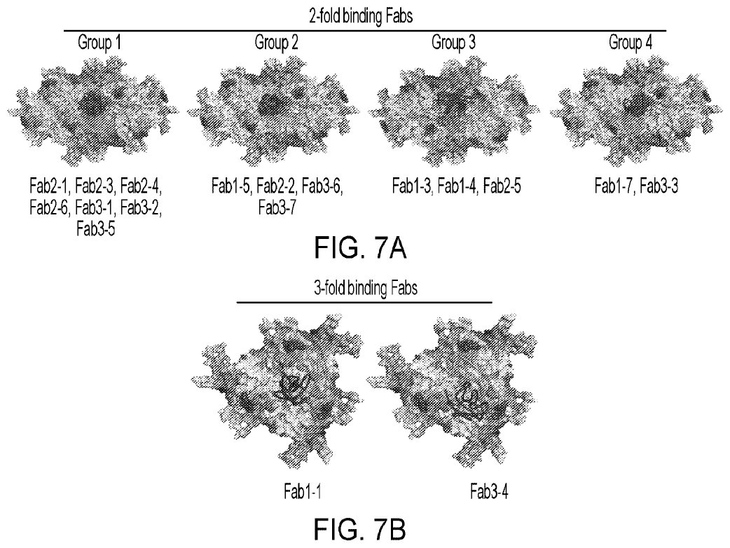

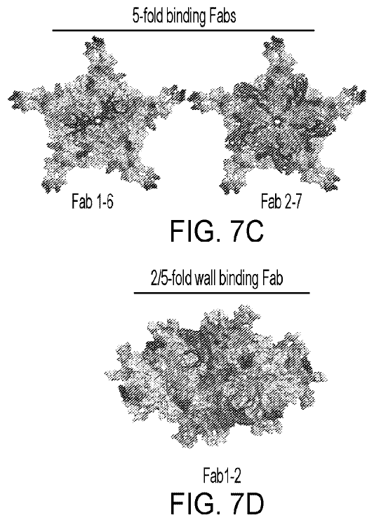

Fabs of interest were classified based on the point at which they bound the AAV9 capsid (at the 2-fold symmetry point, 3-fold symmetry point, 5-fold symmetry point, or the 2/5 fold symmetry wall), as shown in FIGS. 7A-7D. Fabs of each group were further classified, as appropriate, into groups based on their interaction with the AAV9 capsid. A summary of the contacts can be seen in the tables below:

| | | | 1-1 | 1-2 | 1-3 | 1-4 | 1-5 | 1-6 | 1-7 |
|---|---|---|---|---|---|---|---|---|---|
| Heavy | CDR1 | H-bonds | — | S49- | S47- | T50- | S49- | T49- | G45- |
| Chain | | | | Q387: | N457: | Q459: | Q387: | N254: | N709: |
| | | | | 3.2 | 3.2 S47- | 3.5 | 3.3 S50- | 3.3 T49- | 3.1 |
| | | | | S49- | Q458: | T50- | K707: | N668: | Y51- |
| | | | | N709: | 2.5 | T460: 2.8 | 2.7 | 3.0 | D556: |
| | | | | 3.2 | | | | | 2.5 |
| | | | | N50- | | | | | Y52- |
| | | | | Q387: | | | | | T491: 2.8 |
| | | | | 3.1 | | | | | Y52- |
| | | | | | | | | | T492: 3.0 |
| | | salt | — | — | — | — | — | — | — |
| | | bridges | | | | | | | |
| | | other | — | — | G45- | G45- | M47- | — | Y52- |
| | | | | | N457: | Q456: | E712: 3.1 | | R533: 3.3 |
| | | | | | 3.3 F46- | 3.2 | S49- | | |
| | | | | | N457: | Y46- | K707: | | |
| | | | | | 3.4 S50- | N457: | 3.4 S50- | | |
| | | | | | T460: 3.2 | 3.3 | Y706: | | |
| | | | | | F51- | Y52- | 3.1 | | |
| | | | | | V493: | D556: | | | |
| | | | | | 2.4 | 3.2 | | | |
| | CDR2 | H-bonds | K55- | N69- | — | Q81- | R72- | S73- | S75- |
| | | | Q579: | S265: 3.3 | | K707: | Y705: 3.4 | N254: | D532: |
| | | | 2.7 | K71- | | 3.0 | R72- | 2.9 D76- | 3.4 |
| | | | | S261: 2.3 | | | Y706: 3.5 | T672: 2.9 | T92- |
| | | | | Q72- | | | R72- | | N704: |
| | | | | S386: 2.7 | | | K707: | | 3.5 |
| | | | | H78- | | | 3.4 S73- | | S93- |
| | | | | S263: 3.3 | | | D384: | | Y705: |
| | | | | | | | 3.4 | | 3.1/3.3 |
| | | | | | | | S75- | | |
| | | | | | | | E531: 2.8 | | |
| | | | | | | | Y76- | | |
| | | | | | | | D532: | | |
| | | | | | | | 3.0 Y76- | | |
| | | | | | | | R533: 3.2 | | |
| | | salt | — | — | — | D75- | D78- | — | — |
| | | bridges | | | | K557: | R533: 3.7 | | |
| | | | | | | 3.3 | | | |
| | | other | K55- | Q72- | Y72- | — | Y76- | S73- | S73- |
| | | | T593: 2.7 | N709: | D556: | | E531: 2.8 | F670: 2.9 | D532: |
| | | | | 3.2 | 2.5 | | Y76- | G74- | 2.8 |
| | | | | | | | R533: 3.3 | T672: 2.6 | K94- |
| | | | | | | | Y76- | A77- | Y706: |
| | | | | | | | F534: 2.9 | K332: | 3.5 |
| | | | | | | | | 3.0 Y79- | N95- |
| | | | | | | | | G330: | Y706: |
| | | | | | | | | 2.4 V88- | 3.1 |
| | | | | | | | | G330: | |
| | | | | | | | | 3.4 | |
| | CDR3 | H-bonds | N108- | Y120- | S123- | P119- | E126- | W122- | T119- |
| | | | Q597: | D384: | D532: | V493: 3.2 | Y731: | S261: 3.2 | D554: |
| | | | 3.1 | 2.7 | 3.4 | N120- | 2.3 I128- | S123- | 3.0 |
| | | | G109- | Y120- | Y127- | T492: 2.7 | I560: 2.8 | Y274: | R124- |
| | | | Q597: | E529: 3.3 | H527: | V121- | N129- | 2.9 | Q459: |
| | | | 3.0 | Q121- | 3.3 | D556: | A555: 3.0 | Y125- | 2.6 |
| | | | C111- | N270: | Y127- | 3.5 | N129- | R550: 2.4 | |
| | | | W595: | 3.0 | F534: 2.7 | Y124- | V558: 3.1 | | |
| | | | 2.0/3.1 | W123- | | H527: | | | |
| | | | | G266: | | 3.0 | | | |
| | | | | 3.0 | | Y125- | | | |
| | | | | W123- | | K545: | | | |
| | | | | N270: | | 3.3 | | | |
| | | | | 3.0 | | Y125- | | | |
| | | | | | | N562: | | | |
| | | | | | | 3.1 | | | |
| | | | | | | G126- | | | |
| | | | | | | R725: 3.2 | | | |
| | | | | | | G128- | | | |
| | | | | | | R725: 2.9 | | | |
| | | | | | | N129 | | | |
| | | | | | | N704: | | | |
| | | | | | | 2.7/2.7 | | | |
| | | | | | | Y130- | | | |
| | | | | | | E712: 3.4 | | | |
| | | | | | | Y731- | | | |
| | | | | | | D532: | | | |
| | | | | | | 2.6 | | | |
| | | salt | — | — | D130- | — | — | — | — |
| | | bridges | | | K545: | | | | |
| | | | | | 2.7 | | | | |
| | | other | L110- | Y120- | Y127- | P119- | F121- | W122- | V121- |
| | | | W595: | K707: | F535: 3.2 | T492: 3.3 | Y706: | I260: 3.5 | T491: 3.3 |
| | | | 3.4 | 3.3 | G129- | V121- | 2.9 | | V121- |
| | | | C111- | | D556: | T491: 2.8 | M123- | | V493: |
| | | | G594: | | 3.2 | V121- | Y705: 2.9 | | 3.1 |
| | | | 3.2 | | | V493: | F124- | | A122- |
| | | | C111- | | | 3.3 | R391: 2.6 | | T492: 3.4 |
| | | | Q597: | | | Y124- | F124- | | |
| | | | 3.1 | | | D532: | Y731: | | |
| | | | T113- | | | 3.5 | 3.5 | | |
| | | | W595: | | | Y125- | V127- | | |
| | | | 3.1 | | | M559: | I560: 3.1 | | |
| | | | | | | 3.4 | I128- | | |
| | | | | | | Y125- | F535: 3.2 | | |
| | | | | | | P726: 3.5 | I128- | | |
| | | | | | | | N562: | | |
| | | | | | | | 3.3 | | |
| Light | CDR1 | H-bonds | I32- | Y54- | — | G50- | Y52- | S46- | N52- |
| Chain | | | Q588: | N270: | | N704: | D556: | G547: | T494: 3.1 |
| | | | 2.7/3.2 | 2.8 | | 3.4 | 3.1 | 3.0 | |
| | | | | | | | | G50- | |
| | | | | | | | | R550: 2.8 | |
| | | salt | — | — | — | — | — | — | — |
| | | bridges | | | | | | | |
| | | other | Y31- | Y54- | — | S47- | — | S51- | — |
| | | | Q588: | G267: | | Y706: | | R550: 3.2 | |
| | | | 3.3 | 3.5 | | 3.3 | | | |
| | CDR2 | H-bonds | S60- | — | N73- | Y70- | S74- | R74- | — |
| | | | A591: 2.4 | | Y706: | Q495: | Q387: | S469: 2.3 | |
| | | | | | 2.6 | 2.9 | 2.9 S74- | K87- | |
| | | | | | | Y70- | N709: | N552: | |
| | | | | | | R533: 3.3 | 2.4 R76- | 3.4 S88- | |
| | | | | | | Q74- | Q387: | N552: | |
| | | | | | | R533: 3.4 | 2.6 R76- | 3.3 | |
| | | | | | | | K707: | | |
| | | | | | | | 2.9 N82- | | |
| | | | | | | | N270: | | |
| | | | | | | | 3.5 | | |
| | | salt | — | — | — | — | R76- | — | — |
| | | bridges | | | | | D384: | | |
| | | | | | | | 2.4 | | |
| | | other | G61- | L76- | K88- | K87- | F71- | Y70- | — |
| | | | Q590: | Q588: | Y706: | Y706: | Y706: | T264: 3.2 | |
| | | | 3.1 | 3.0 | 3.2 | 3.2 | 3.0 N75- | K87- | |
| | | | | | | | Y706: | D551: | |
| | | | | | | | 3.0 N75- | 3.2 D89- | |
| | | | | | | | S708: 3.3 | D551: | |
| | | | | | | | G79- | 2.7 D89- | |
| | | | | | | | G530: | N552: | |
| | | | | | | | 2.9 | 3.5 | |
| | CDR3 | H-bonds | W95- | S113- | — | D114- | S117- | R115- | W112- |
| | | | Q588: | G267: | | N704: | T492: 2.8 | D657 | T494: 2.8 |
| | | | 2.4 | 3.0 | | 3.3 | | 2.3 R115- | N117- |
| | | | D96- | Y114- | | | | S669: 3.5 | Q495: |
| | | | Q588: | G267: | | | | | 2.7 |
| | | | 3.2 | 3.2 | | | | | |
| | | | S97- | Y114- | | | | | |
| | | | Q588: | S268: 2.4/ | | | | | |
| | | | 3.3 | 3.0 | | | | | |
| | | | | S115- | | | | | |
| | | | | S268: 3.3 | | | | | |
| | | | | Y118- | | | | | |
| | | | | S265: 3.3 | | | | | |
| | | | | Y118- | | | | | |
| | | | | G266: | | | | | |
| | | | | 3.5 | | | | | |
| | | salt | — | — | — | — | — | — | — |
| | | bridges | | | | | | | |
| | | other | — | S113- | S115- | S115- | — | R115- | — |
| | | | | G266: | Y706: | Y706: | | P658: 3.1 | |
| | | | | 3.2 | 3.1 | 3.1 | | R115- | |
| | | | | T115- | S116- | | | P659: 3.4 | |
| | | | | S265: 3.3 | K707: | | | | |
| | | | | | 3.4 | | | | |

| | | | | | | | |
|---|---|---|---|---|---|---|---|
| total | 18 | 26 | 17 | 33 | 39 | 27 | 20 |
| contacts | | | | | | | |
| interface | 1307 | 1734 | 1347 | 1778 | 2177 | 1779 | 1272 |
| area (Å²) | | | | | | | |
| # VPs | 2 | 4 | 3 | 4 | 4 | 3 | 2 |
| contacted | | | | | | | |

| | | | 2-1 | 2-2 | 2-3 | 2-4 | 2-5 | 2-6 | 2-7 |
|---|---|---|---|---|---|---|---|---|---|
| Heavy | CDR1 | H-bonds | S49- | S49- | — | N50- | G45- | S49- | — |
| Chain | | | Y705: | K707: | | N704: | N457: | Y705: | |
| | | | 2.8 | 3.2 | | 3.4 | 3.4 S50- | 2.5 | |
| | | | | | | | Q459: | Y51- | |
| | | | | | | | 2.9 | N704: | |
| | | | | | | | | 3.2 | |
| | | salt | — | — | — | — | — | — | — |
| | | bridges | | | | | | | |
| | | other | S49- | S49- | S49- | P47- | Y51- | G45- | — |
| | | | Y706: | Y706: 3.4 | Y706: 3.2 | N704: | V493: | Y706: | |
| | | | 3.4 | | | 3.5 | 3.4 | 3.3 | |
| | | | | | | | | S47- | |
| | | | | | | | | Y706: | |
| | | | | | | | | 3.3 | |
| | CDR2 | H-bonds | S71- | Y71- | T76- | S73- | S71- | S71- | S71- |
| | | | D532: | G530: | G530: 3.4 | D532: | D556: | D532: | E718: 3.0 |
| | | | 2.5 S73- | 3.4 Y71- | Y78- | 3.4 G75- | 2.6 S73- | 2.9 | S73- |
| | | | D532: | D532: | Q495: 2.9 | G530: | D556: | T76- | T717: 3.1 |
| | | | 2.5 S76- | 2.6 Y72- | Y78- | 2.7 | 2.9 Y76- | D532: | |
| | | | D532: | D384: | R533: 3.1 | G75- | K545: | 2.8 | |
| | | | 2.7 N78- | 2.4 S73- | | D532: | 3.0 | N93- | |
| | | | H₂O- | G530: | | 2.9 S76- | | Y706: | |
| | | | T491 | 2.5 N75- | | D532: | | 3.5 | |
| | | | 3.0/3.1 | N495: | | 3.3 | | | |
| | | | | 3.3 N75- | | | | | |
| | | | | R533: 3.3 | | | | | |
| | | salt | — | — | — | — | — | — | — |
| | | bridges | | | | | | | |
| | | other | G75- | Y72- | T76- | Y93- | S73- | G74- | S76- |
| | | | G530: | G530: | R333: 3.5 | Y706: | D554: | G530: | E718: 3.4 |
| | | | 3.4 | 3.0 Y72- | N93- | 3.5 Y93- | 3.1 | 3.4 | |
| | | | | K707: | Y706: 3.2 | K707: | | | |
| | | | | 3.5 T92- | | 3.1 | | | |
| | | | | Q387: | | | | | |
| | | | | 3.1 | | | | | |
| | CDR3 | H-bonds | Y121- | Y121- | R124- | G124- | T121- | L124- | G127- |
| | | | N704: | K567: | N562: 3.0 | N562: | T491: 2.9 | N562: | D327: |
| | | | 3.5 | 3.5 | R124- | 2.6 E125- | Y126- | 3.1 | 3.2 |
| | | | R124- | G122- | H₂O- | N562: | K545: | G126- | S128- |
| | | | N562: | D532: | H527 | 3.2 E125- | 2.6 | N562: | E324: 3.1 |
| | | | 2.9 | 3.3 | 2.9/3.0 | Y731: | N125- | 3.0 | S128- |
| | | | S125- | G122- | E125- | 3.3 I127- | I560: 3.0 | Y127- | T672: |
| | | | N562: | N562: | N562: 2.7 | I560: 2.7 | G132- | S526: 3.4 | 3.1/3.4 |
| | | | 3.3 Y127- | 2.4 | E125- | Y131- | R533: 2.8 | F128- | T130- |
| | | | T561: 3.0 | Y124- | G728: 3.3 | T492: 2.9 | | T491: 3.3 | F670: |
| | | | F128- | H527: | E125- | | | F128- | 2.7/2.9 |
| | | | D532: | 3.5 | H₂O-R730 | | | D532: | T130- |
| | | | 2.9 | Y124- | 2.7/3.5 | | | 3.3 | T672: 2.4 |
| | | | Y132- | F534: 3.3 | E125- | | | | T131- |
| | | | D556 3.2 | Y124- | Me²⁺- | | | | S669: 3.5 |
| | | | | H₂O- | E565- | | | | N132- |
| | | | | S540 | N562 | | | | N254: |
| | | | | 2.8/2.5 | 2.3/2.6/2.6 | | | | 2.7/3.1 |
| | | | | | I127- | | | | N132- |
| | | | | | I560: 2.9 | | | | N668: |
| | | | | | Y131- | | | | 2.9 |
| | | | | | T492: 3.3 | | | | Y134- |
| | | | | | | | | | K258: |
| | | | | | | | | | 3.2 |
| | | salt | D123- | -— | D123- | E125- | — | D123- | |
| | | bridges | R725: 2.6 | | R725: 3.4 | R725: 3.2 | | R725: 2.6 | |
| | | | R124- | | R124- | | | | |
| | | | E529: 3.9 | | D564: 3.4 | | | | |
| | | | R124- | | | | | | |
| | | | D564: | | | | | | |
| | | | 3.3 | | | | | | |
| | | other | Y121- | Y121- | R124- | E125- | T120- | Y122- | I129- |
| | | | N704: | D532: | Y705: 2.8 | P726: 2.9 | T492: 3.2 | D532: | A656: |
| | | | 3.2 | 2.7 | E125- | V126- | T120- | 3.4 | 3.5 I129- |
| | | | R124- | Y124- | P726: 3.2 | I560: 3.2 | R533: 3.4 | L124- | S669: 2.7 |
| | | | R391: 2.7 | D532: | V126- | V126- | Y122- | D564: | I129- |
| | | | Y127- | 2.6 | I560: 3.1 | P726: 3.4 | T491: 3.5 | 3.4 Y126- | F670: 3.3 |
| | | | F534: 3.3 | | V126- | I127- | Y122- | D532: | T131- |
| | | | Y127- | | P726: 3.4 | D532: | R533: 3.2 | 3.2 | N668: |
| | | | F535: 3.3 | | I127- | 3.3 I127- | N125- | Y126- | 3.5 |
| | | | Y127- | | D532: 3.2 | N562: | A555: 3.4 | F534: 3.2 | |
| | | | I560: 3.5 | | I127- | 3.3 | N125- | Y126- | |
| | | | F128- | | R533: 3.5 | K129- | V558: 3.2 | N562: | |
| | | | T491: 3.0 | | R128- | T491: 3.2 | W128- | 2.5 | |
| | | | W129- | | T491: 3.4 | | P726: 3.2 | L129- | |
| | | | T491: 3.5 | | A129- | | | T491: 3.3 | |
| | | | W129- | | T491: 3.0 | | | | |
| | | | A555: | | | | | | |
| | | | 3.3 | | | | | | |
| Light | CDR1 | H-bonds | Y53- | — | Q49- | — | Y53- | — | N45- |
| Chain | | | D556: | | N457: 3.4 | | D532: | | N552: |
| | | | 2.9 | | N52- | | 2.7 | | 2.9 |
| | | | | | D554: 3.4 | | | | |
| | | salt | — | — | — | — | — | — | — |
| | | bridges | | | | | | | |
| | | other | — | A51- | — | Q47- | — | — | — |
| | | | | D556: | | N457: | | | |
| | | | | 3.2 Y52- | | 3.2 | | | |
| | | | | D556: | | | | | |
| | | | | 3.1 | | | | | |
| | CDR2 | H-bonds | — | S74- | S78- | — | — | — | — |
| | | | | Q387: | Q387: 3.2 | | | | |
| | | | | 2.9 N75- | S78- | | | | |
| | | | | S708: 3.3 | N709: 3.2 | | | | |
| | | | | R76- | | | | | |
| | | | | K707: | | | | | |
| | | | | 3.1 R76- | | | | | |
| | | | | Q387: | | | | | |
| | | | | 2.7 S78- | | | | | |
| | | | | Y705: | | | | | |
| | | | | 2.5 S78- | | | | | |
| | | | | K707: | | | | | |
| | | | | 2.8 G80- | | | | | |
| | | | | G530: | | | | | |
| | | | | 2.8 N83- | | | | | |
| | | | | D384: | | | | | |
| | | | | 3.2 N83- | | | | | |
| | | | | G385: | | | | | |
| | | | | 3.5 | | | | | |
| | | salt | — | R76- | — | — | D71- | — | — |
| | | bridges | | D384: | | | R533: 2.9 | | |
| | | | | 2.6 | | | | | |
| | | other | S77- | S74- | — | — | K87- | — | — |
| | | | Q387: | N709: | | | Y706: | | |
| | | | 3.4 | 3.4 N75- | | | 3.3 | | |
| | | | | K707: | | | | | |
| | | | | 3.4 S79- | | | | | |
| | | | | D532: | | | | | |
| | | | | 3.0 | | | | | |
| | CDR3 | H-bonds | — | S118- | — | Y114- | — | — | — |
| | | | | V493: 3.4 | | Q459: | | | |
| | | | | | | 2.7 | | | |
| | | | | | | Y114- | | | |
| | | | | | | T460: 3.4 | | | |
| | | | | | | S115- | | | |
| | | | | | | T494: 3.2 | | | |
| | | salt | — | — | — | — | — | — | — |
| | | bridges | | | | | | | |
| | | other | D114- | S118- | — | Y114- | S114- | — | — |
| | | | V493: | T492: 3.5 | | V493: | Y706: | | |
| | | | 3.4 | | | 3.4 | 3.4 | | |
| | | | D114- | | | A116- | S115- | | |
| | | | V494: | | | T494: 2.8 | Y706: | | |
| | | | 3.5 | | | | 3.4 | | |
| | | | S117- | | | | | | |
| | | | T494: 3.5 | | | | | | |

| | | | | | | | |
|---|---|---|---|---|---|---|---|
| total | 29 | 36 | 28 | 26 | 22 | 20 | 20 |
| contacts | | | | | | | |
| interface | 1630 | 1587 | 1567 | 1448 | 1347 | 1494 | 987 |
| area (Å²) | | | | | | | |
| # VPs | 4 | 4 | 4 | 3 | 3 | 2 | 2 |
| contacted | | | | | | | |

| | | | 3-1 | 3-2 | 3-3 | 3-4 | 3-5 | 3-6 | 3-7 |
|---|---|---|---|---|---|---|---|---|---|
| Heavy | CDR1 | H-bonds | S49- | S49- | S47- | — | T47- | S49- | S49- |
| Chain | | | Y705: | Y705: | Y706: | | N704: 2.7 | Q387: | K707: |
| | | | 2.8 R50- | 2.9 | 2.9 | | | 2.8 S50- | 2.5 |
| | | | N704: | | S50- | | | K707: | N50- |
| | | | 2.8 | | K545: | | | 3.1 | K707: |
| | | | | | 2.8 S50- | | | | 3.3 |
| | | | | | P724: 3.3 | | | | |
| | | | | | S52- | | | | |
| | | | | | K545: | | | | |
| | | | | | 3.5 | | | | |
| | | | | | Y54- | | | | |
| | | | | | T492: | | | | |
| | | | | | 3.1/2.8 | | | | |
| | | salt | — | — | H53- | — | — | E53- | — |
| | | bridges | | | D556: | | | R533: 3.3 | |
| | | | | | 3.2 | | | | |
| | | other | S49- | S49- | Y54- | G45- | G45- | H47- | N50- |
| | | | Y706: | Y706: | T491: 3.3 | Q588: | Y706: 3.4 | Y706: | Y706: |
| | | | 2.8 | 3.3 | | 3.0 | F46- | 3.4 S49- | 3.0 |
| | | | | | | F46- | Y706: 3.4 | K707: | |
| | | | | | | Q588: | | 3.1 S50- | |
| | | | | | | 3.4 | | Y706: 3.3 | |
| | CDR2 | H-bonds | S73- | S71- | S71- | — | S71- | Y69- | Y72- |
| | | | G530: | D532: | R533: 2.7 | | D532: 3.0 | R533: 2.7 | G530: |
| | | | 3.4 | 3.2 | S77- | | G73- | S73- | 3.4 |
| | | | | | D532: | | D532: 3.0 | K707: | S73- |
| | | | | | 3.2 | | T75- | 2.8 T75- | G530: |
| | | | | | S77- | | D532: 2.5 | G530: | 3.1 |
| | | | | | R533: 3.3 | | Y77- | 3.3 Y76- | S75- |
| | | | | | Y79- | | Q495: 2.9 | D532: | R533: 2.8 |
| | | | | | R533: 2.9 | | Y77- | 3.0 Y76- | |
| | | | | | | | R533: 3.4 | R533: 3.0 | |
| | | | | | | | N92- | N93- | |
| | | | | | | | N704: 3.5 | Q387: | |
| | | | | | | | | 3.3 P94- | |
| | | | | | | | | N709: | |
| | | | | | | | | 3.1 | |
| | | salt | — | — | — | — | — | — | — |
| | | bridges | | | | | | | |
| | | other | S71- | N93- | Y73- | F76- | S72- | S73- | Y72- |
| | | | R533: 3.1 | Y706: | D532: | S499: 3.4 | Y706: 3.3 | G530: | D384: |
| | | | Y78- | 3.3 | 3.1 | F76- | N92- | 3.2 | 3.3 |
| | | | T492: 3.3 | | L75- | Q590: | Y706: 3.4 | Y76- | |
| | | | | | S532: 2.4 | 3.3 | | E531: 3.1 | |
| | | | | | L75- | F76- | | | |
| | | | | | N562: | A591: 3.1 | | | |
| | | | | | 2.6 | | | | |
| | | | | | S95- | | | | |
| | | | | | Y706: | | | | |
| | | | | | 2.9 | | | | |
| | | | | | N97- | | | | |
| | | | | | Y706: | | | | |
| | | | | | 3.4 | | | | |
| | CDR3 | H-bonds | V124- | Y121- | — | T125- | C121- | E123- | S120- |
| | | | N562: | N704: | | W595: | R725: 2.5 | N704: | Y705: |
| | | | 3.5 | 3.2 | | 3.5 | D124- | 2.6 | 2.7 |
| | | | T125- | L124- | | T125- | D562: 3.0 | P125- | S120- |
| | | | N562: | N562: | | Q597: | D124- | N562: | Y706: |
| | | | 2.4 I127- | 3.1 | | 3.5 | G728: 2.6 | 3.1 | 3.1 |
| | | | I560: 2.7 | S125- | | T126- | D124- | Y126- | Y122- |
| | | | V128- | P726: 3.5 | | N598: | Y731: 3.3 | F534: 2.5 | T702: |
| | | | T491: 3.3 | G126- | | 3.5 | D124- | Y128- | 2.9 |
| | | | | N562: | | W128- | Me²⁺- | D532: | Y122- |
| | | | | 3.4 | | W595: | E565- | 2.3 | N704: |
| | | | | Y127- | | 2.4/2.6 | N562- | Y129- | 3.4 |
| | | | | S526: 3.3 | | R129- | D611 | N704: | Y122- |
| | | | | Y127- | | T593: 2.6 | 2.4/3.1/3.2 | 2.9 | Y705: |
| | | | | T561: 3.4 | | | Y126- | | 3.2 |
| | | | | G130- | | | F534: 2.9 | | N123- |
| | | | | T492: 2.9 | | | Y126- | | N562: |
| | | | | Y132- | | | I560: 2.8 | | 3.0 |
| | | | | D556: | | | | | N123- |
| | | | | 2.9 | | | | | E564: 2.9 |
| | | | | Y137- | | | | | E124- |
| | | | | Y706: | | | | | N562: |
| | | | | 2.8 | | | | | 3.0 |
| | | | | | | | | | E124- |
| | | | | | | | | | G728: |
| | | | | | | | | | 3.0 |
| | | | | | | | | | E124- |
| | | | | | | | | | Y731: |
| | | | | | | | | | 2.5 |
| | | | | | | | | | I126- |
| | | | | | | | | | I560: 3.0 |
| | | salt | D122- | D123- | — | — | — | — | — |
| | | bridges | R533: 3.2 | R725: 3.2 | | | | | |
| | | | E123- | R131- | | | | | |
| | | | R725: 2.8 | D556: | | | | | |
| | | | | 2.9 | | | | | |
| | | other | T125- | Y121- | — | T125- | D124- | F121- | R119- |
| | | | P726: 3.1 | R725: 3.4 | | N598: | T561: 3.1 | Y706: | N704: |
| | | | M126- | Y122- | | 3.0 T126- | C125- | 3.4 | 3.0 |
| | | | I560: 3.3 | Y705: | | W595: | I560: 2.8 | E123- | R119- |
| | | | M126- | 3.4 | | 3.3 | C125- | Y705: | Y706: |
| | | | P724: 3.0 | L124- | | T126- | P726: 3.4 | 3.1 | 3.2 |
| | | | M126- | E564: 3.4 | | V596: | Y126- | P125- | Y122- |
| | | | R725: 2.7 | Y127- | | 2.8 | S526: 3.0 | E564: 2.4 | S703: 3.2 |
| | | | M126- | D532: | | T126- | Y126- | Y126- | E124- |
| | | | P726: 3.2 | 3.1 | | N598: | H527: 3.1 | D532: | P726: 2.4 |
| | | | I127- | Y127- | | 2.8/2.8 | Y126- | 3.0 | V125- |
| | | | D532: | F534: 3.1 | | S127- | D532: 2.8 | Y126- | I560: 3.2 |
| | | | 3.2 I127- | Y127- | | V580: | Y126- | F535: 3.2 | V125- |
| | | | N562: | F535: 3.4 | | 3.4 | F535: 3.4 | Y126- | P726: 3.5 |
| | | | 2.9 | Y127- | | S127- | Y126- | I560: 3.5 | I126- |
| | | | V128- | N562: | | W595: | D562: 2.7 | Y126- | D532: |
| | | | R533: 2.9 | 2.7 | | 3.3 S127- | | N562: | 2.9 |
| | | | M131- | Y128- | | Q597: | | 2.9 | I126- |
| | | | D556: | T491: 2.9 | | 2.9 | | | D562: |
| | | | 3.1 | G130- | | W128- | | | 2.9 |
| | | | | T491: 3.1 | | T582: 3.0 | | | |
| | | | | | | W128- | | | |
| | | | | | | Q597: | | | |
| | | | | | | 3.4 | | | |
| | | | | | | W132- | | | |
| | | | | | | Q592: | | | |
| | | | | | | 2.9 | | | |
| Light | CDR1 | H-bonds | — | — | N52- | — | Q49- | — | Y54- |
| Chain | | | | | N498: | | N459: 3.3 | | D556: |
| | | | | | 2.8 S53- | | | | 2.9 |
| | | | | | Q458: | | | | |
| | | | | | 3.1 | | | | |
| | | | | | W54- | | | | |
| | | | | | Q459: | | | | |
| | | | | | 2.8 | | | | |
| | | salt | — | — | — | — | R53- | — | — |
| | | bridges | | | | | D554: 3.8 | | |
| | | other | — | — | — | — | — | — | — |
| | CDR2 | H-bonds | — | T78- | S72- | N74- | — | S74- | S74- |
| | | | | Q387: | Q458: | Q590: | | Q387: | Q387 |
| | | | | 3.2 | 2.3 | 2.9 | | 2.9 S74- | 2.9 S74- |
| | | | | | | | | N709: | N709: |
| | | | | | | | | 3.5 R76- | 3.4 N75- |
| | | | | | | | | K707: | K707: |
| | | | | | | | | 3.0/3.2 | 3.2 |
| | | | | | | | | S78- | R76- |
| | | | | | | | | D532: | Q387: |
| | | | | | | | | 3.5 S78- | 2.6 R76- |
| | | | | | | | | K707: | K707: |
| | | | | | | | | 3.4 N82- | 3.3 N82- |
| | | | | | | | | G385: | D384: |
| | | | | | | | | 3.2 | 2.8 |
| | | salt | — | — | — | — | — | — | R76- |
| | | bridges | | | | | | | D384: |
| | | | | | | | | | 2.6 |
| | | other | — | — | — | — | — | N75- | N75- |
| | | | | | | | | Y706: | S708: 2.3 |
| | | | | | | | | 2.5 R76- | G79- |
| | | | | | | | | D384: | G530: |
| | | | | | | | | 3.3 G79- | 3.5 N82- |
| | | | | | | | | G530: | G385: |
| | | | | | | | | 3.0 | 3.0 |
| | CDR3 | H-bonds | Y115- | — | A113- | S116- | Y114- | S117- | — |
| | | | N457: | | T494: 3.3 | Q588: | N457: 3.3 | T492: 3.5 | |
| | | | 3.5 | | N114- | 2.9 | Y114- | R115- | |
| | | | Y115- | | T494: 2.7 | S116- | Q459: | N457: | |
| | | | Q459: | | | Q590: | 2.9/3.0 | 3.4 | |
| | | | 2.3 | | | 2.6 | I116- | R115- | |
| | | | W116- | | | | T494: | Q459: | |
| | | | T492: 3.1 | | | | 3.3/3.4 | 2.9 | |
| | | | W116- | | | | I116- | | |
| | | | T494: 3.0 | | | | Q495: 3.4 | | |
| | | salt | — | — | — | — | — | — | — |
| | | bridges | | | | | | | |
| | | other | P117- | — | F116- | — | Y114- | — | — |
| | | | T494: 2.9 | | Q495: | | T494: 3.0 | | |
| | | | | | 3.1 | | S115- | | |
| | | | | | | | T494: 3.3 | | |

| | | | | | | | |
|---|---|---|---|---|---|---|---|
| total | 26 | 25 | 24 | 25 | 36 | 40 | 37 |
| contacts | | | | | | | |
| interface | 1497 | 1367 | 1320 | 1280 | 1452 | 2068 | 2001 |
| area (Å²) | | | | | | | |
| # VPs | 3 | 3 | 2 | 3 | 3 | 5 | 4 |
| contacted | | | | | | | |

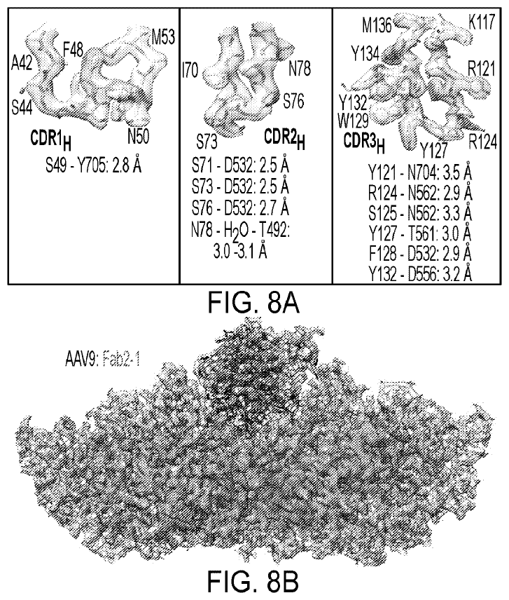

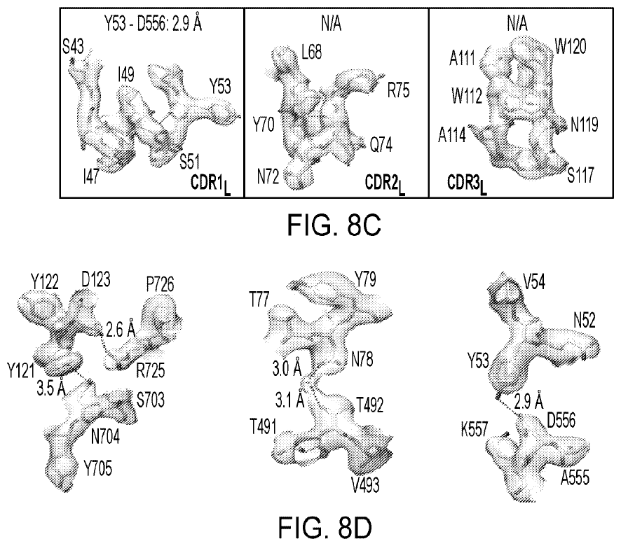

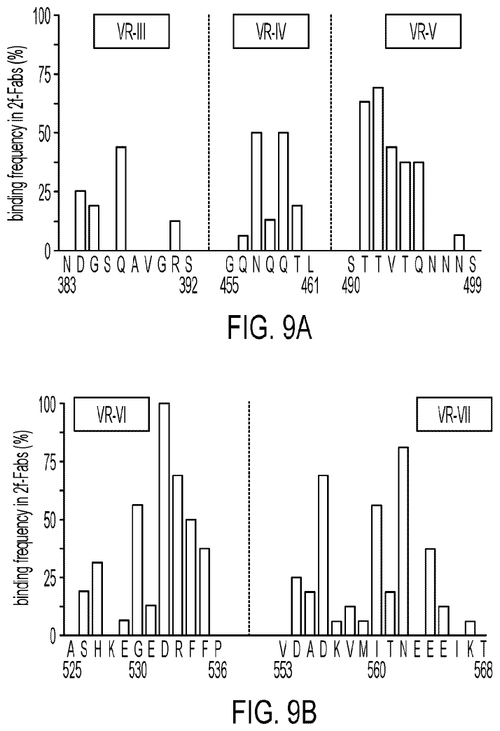

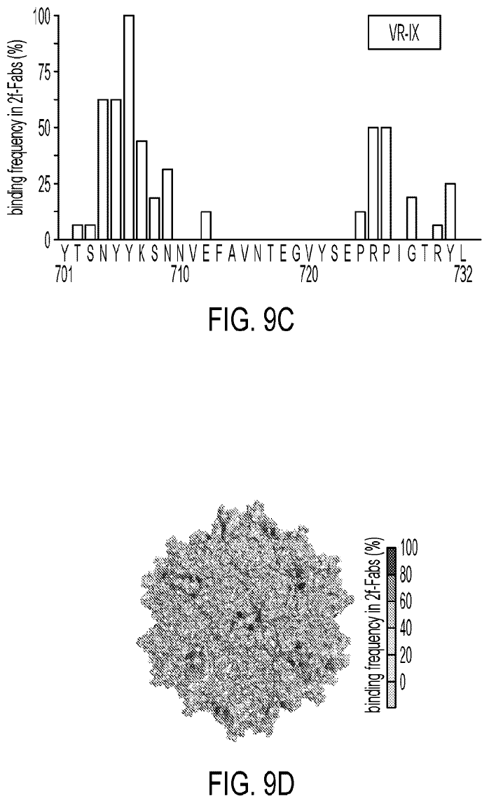

The contacts of Fab2-1 to the AAV9 capsid were determined as shown in FIGS. 8A-8D. FIGS. 9A-9D show the contact frequency of Fabs binding the 2-fold symmetry point. FIG. 9A shows the percentage of contacted capsids by residue within variable regions III, IV, and V, while FIG. 9B shows the percentage of contacted capsids by residue within variable regions VI and VII, and FIG. 9C shows the percentage of contacted capsids by residue within variable region IX. FIG. 9D shows a heatmap of binding frequency.

### Fab Induced Structural Rearrangement of the AAV9 Capsid.

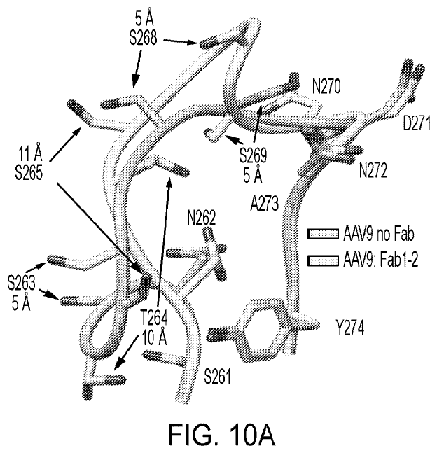

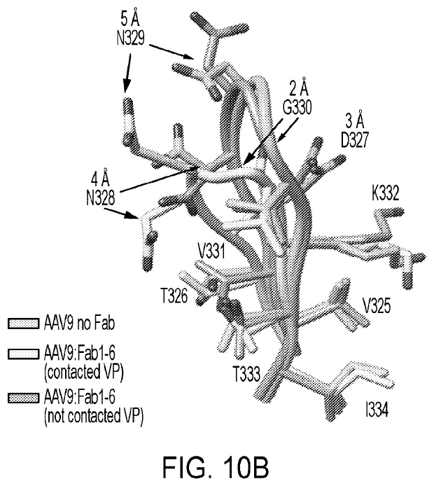

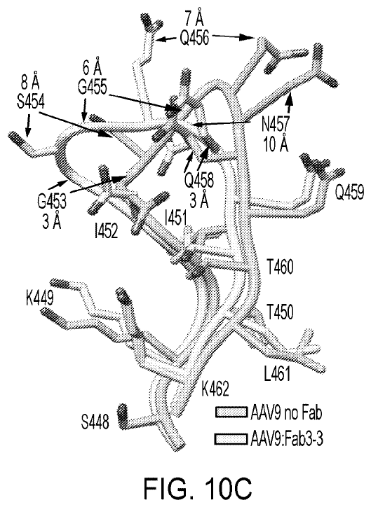

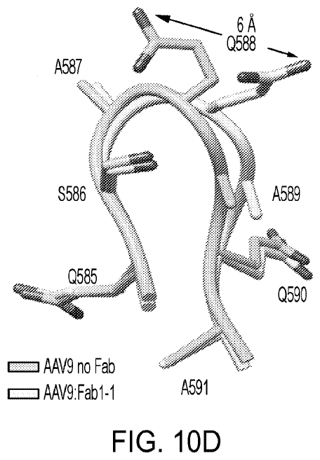

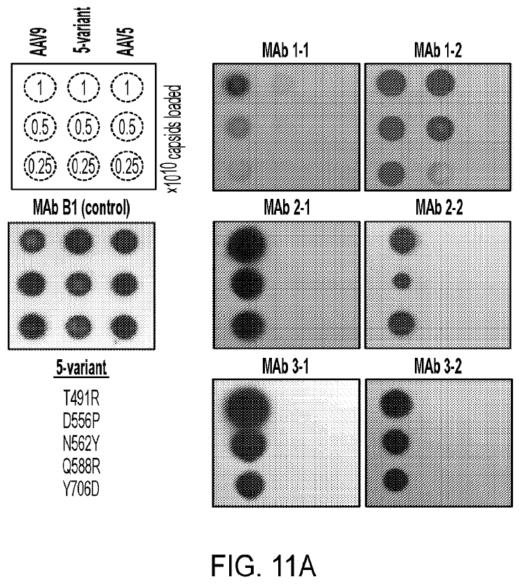

Cryo-EM showed that binding of Fab1-2 to the AAV9 capsid leads to an alternative conformation of VR-I. The conformation in absence of the antibody and in presence of Fab1-2 are shown in FIG. 10A as ribbon diagrams with amino acid side chains. Movements of ≥3 Å between the indicated amino acids are indicated. Similarly, binding of Fab1-6 lead to an alternative conformation of VR-II (FIG. 10B), binding of Fab3-3 led to an alternative conformation of VR-II (FIG. 10C), and binding of Fab1-1 led to an alternative conformation of VR-III.

### Example 5: Antibody Escape Capsid Variants

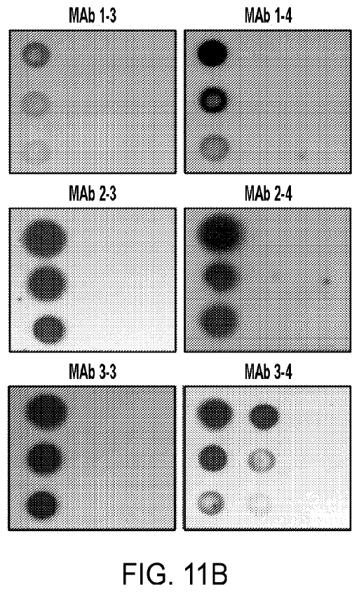

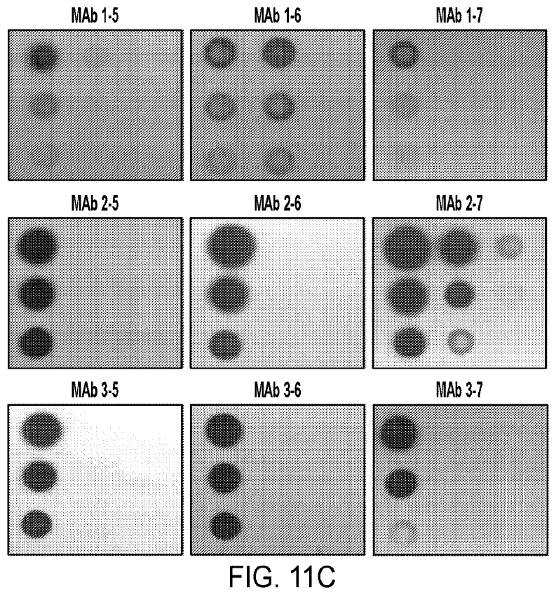

Based on data regarding binding of neutralizing antibodies to the AAV9 capsid, variant AAV9 capsids were designed to evade said antibodies. The 21 human MAbs described in Example 2 were tested against a wild-type AAV9 capsid (positive control), a wild-type AAV5 capsid (negative control), and a non-limiting example of an engineered AAV9 capsid variant (5-variant). The non-limiting example of an engineered AAV9 capsid variant included the five amino acid substitutions described in the lower left corner of FIG. 11A (T491R, D556P, N562Y, Q588R, Y706D). The dot blots in FIGS. 11A-C show the results. The engineered AAV9 capsid variant evaded 17 of the 21 MAbs, all but MAb1-2, 1-6, 2-7, and 3-4.

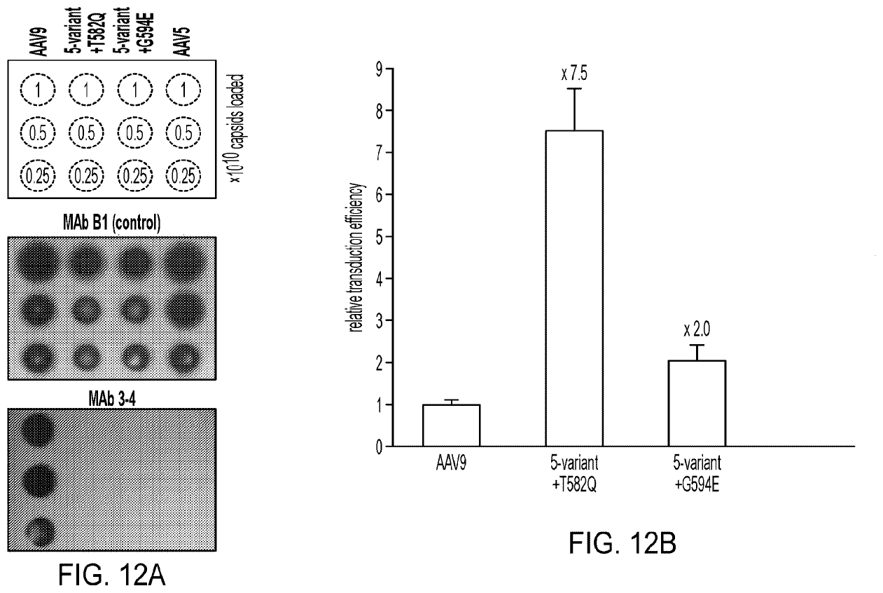

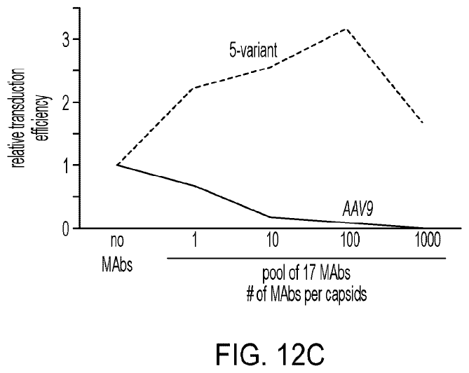

Further amino acid substitutions were introduced to the AAV9 variant capsid, to produce a capsid variant that evaded more than 17 of the 21 MAbs. As shown in FIG. 12A, further adding substitution T582Q or G594E allowed escape from Mab3-4. An infectivity analysis showed that both AAV9 variants have enhanced transduction compared to wild-type AAV9. In the presence of a pooled MAb solution containing equal amounts of the 17 different MAbs, rAAV particles with 5-variant capsids showed similar levels of transduction, relative to transduction in the absence of the pooled MAb solution (FIG. 12C).

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be illustrative examples, and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited. In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g, “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

We claim:

1. A recombinant adeno-associated virus 9 (rAAV9) particle comprising a capsid protein comprising one or more mutations, wherein said mutations result in modulated reactivity to a neutralizing antibody, altered or unchanged production efficiency, and/or altered or unchanged infectivity, relative to a wild-type AAV9 particle having a capsid protein with an amino acid sequence of SEQ ID NO: 2.

2. The rAAV9 particle of claim 1, wherein the neutralizing antibody comprises one or more Fragment antigen-binding (Fab) region(s) which binds AAV9.

3. The rAAV9 particle of claim 1 or 2, wherein the neutralizing antibody is against AAV9.

4. The rAAV9 particle of claim 1 or 2, wherein the neutralizing antibody is against AAV1, AAV2, AAV2.5, AAV3, AAV4, AAV5, AAV6, AAV7, AAV8, AAVrh.10, AAVrh,74, AAV10, AAV11, AAV12, AAV-DJ, PHP.eB, AvianAAV, or AAV13.

5. The rAAV9 particle of any one of claims 1-4, wherein the neutralizing antibody is Immunoglobulin G (IgG), Immunoglobulin M (IgM), Immunoglobulin A (IgA), Immunoglobulin D (IgD), or Immunoglobulin E (IgE).

6. The rAAV9 particle of claim 5, wherein the neutralizing antibody is IgG.

7. The rAAV9 particle of any one of claims 1-6, wherein the neutralizing antibody is a pre-existing neutralizing antibody against AAV9 or is generated in a subject in response to administration of an AAV vector, optionally an AAV9 vector.

8. The rAAV9 particle of claim 7, wherein the AAV vector comprises a nucleic acid sequence encoding a therapeutic molecule.

9. The rAAV9 particle of claim 7 or 8, wherein the neutralizing antibody is isolated from the subject.

10. The rAAV9 particle of any one of claims 1-9, wherein the neutralizing antibody comprises one or more Fragment antigen-binding (Fab) region(s) which binds AAV9.

11. The rAAV9 particle of any of the preceding claims, wherein:
(a) the reactivity to neutralizing antibodies is decreased; and/or
(b) the production efficiency is:
(i) unchanged, or
(ii) increased; and/or
(c) the infectivity is:
(i) unchanged, or
(ii) increased.

12. The rAAV9 particle of claim 11, wherein:
(a) the reactivity to neutralizing antibodies is decreased by 20-100%; and/or
(b) the production efficiency is:
(i) unchanged, or
(ii) increased by 50-400%; and/or
(c) the infectivity is:
(i) unchanged, or
(ii) increased by 50-400%.

13. The rAAV9 particle of claim 11 or 12, wherein the reactivity to neutralizing antibodies is decreased by 75-100%.

14. The rAAV9 particle of any one of the preceding claims, wherein the capsid protein is one or more of the capsid proteins selected from the group consisting of: VP1, VP2 and VP3.

15. The rAAV9 particle of any one of the preceding claims, wherein the one or more mutations comprise an amino acid substitution or an amino acid deletion.

16. The rAAV9 particle of any one of the preceding claims, wherein the one or more mutations are at amino acid positions selected from the group consisting of: D327, N329, D441, T491, V493, R533, N552, V553, D556, 1560, N562, Q588, W595, Q597, A656, P658, Y706, and N709.

17. The rAAV9 particle of any one of the preceding claims, wherein the one or more mutations are amino acid substitution(s) selected from the group consisting of: D327R, D327T, N329D, D441N, T491R, V493P, R533S, N552A, V553A, D556P, 1560F, N562Y, Q588R, W595G, Q597S, A656G, P658L, Y706D, N709I, and N709Y.

18. The rAAV9 particle of claim 17, wherein the one or more mutations is D327R.

19. The rAAV9 particle of claim 17, wherein the one or more mutations is D441N.

20. The rAAV9 particle of claim 17, wherein the one or more mutations is T491R.

21. The rAAV9 particle of claim 17, wherein the one or more mutations is V493P.

22. The rAAV9 particle of claim 17, wherein the one or more mutations is R533S.

23. The rAAV9 particle of claim 17, wherein the one or more mutations is N552A.

24. The rAAV9 particle of claim 17, wherein the one or more mutations is V553A.

25. The rAAV9 particle of claim 17, wherein the one or more mutations is D556P.

26. The rAAV9 particle of claim 17, wherein the one or more mutations is I560F.

27. The rAAV9 particle of claim 17, wherein the one or more mutations is N562Y.

28. The rAAV9 particle of claim 17, wherein the one or more mutations is Q588R.

29. The rAAV9 particle of claim 17, wherein the one or more mutations is Y706D.

30. The rAAV9 particle of claim 17, wherein the one or more mutations is N709I.

31. The rAAV9 particle of claim 17, wherein the one or more mutations is N709Y.

32. The rAAV9 particle of claim 17, wherein the one or more mutations are D327T and N329D.

33. The rAAV9 particle of claim 17, wherein the one or more mutations are N552A and V553A.

34. The rAAV9 particle of claim 17, wherein the one or more mutations are W595G and Q597S.

35. The rAAV9 particle of claim 17, wherein the one or more mutations are A656G and P658L.

36. The rAAV9 particle of claim 17, wherein the one or more mutations are Y706D and N709I.

37. The rAAV9 particle of claim 17, wherein the one or more mutations are Q588R and T491R.

38. The rAAV9 particle of any one of the preceding claims, further comprising a transgene comprising a gene of interest.

39. The rAAV9 particle of claim 38, wherein the gene of interest comprises a survival motor neuron (SMN) gene.

40. The rAAV9 particle of claim 39, wherein the SMN gene is a SMN1 or SMN2 gene.

41. The rAAV9 particle of any one of claims 38-40, wherein the gene of interest encodes a therapeutic protein.

42. The rAAV9 particle of claim 41, wherein the therapeutic protein is an antibody, a peptibody, a growth factor, a clotting factor, a hormone, a membrane protein, a cytokine, a chemokine, an activating or inhibitory peptide acting on cell surface receptors or ion channels, a cell-permeant peptide targeting intracellular processes, a thrombolytic, an enzyme, a bone morphogenetic protein, a nuclease or other protein used for gene editing, an Fc-fusion protein, or an anticoagulant.

43. The rAAV9 particle of claim 41 or 42, wherein the therapeutic protein is a survival motor neuron (SMN) protein.

44. The rAAV9 particle of any one of claims 38-43, wherein the gene of interest encodes a detectable molecule.

45. The rAAV9 particle of claim 44, wherein the detectable molecule is a fluorescent protein, a bioluminescent protein, or a protein that provides color, or a fragment thereof.

46. A composition comprising a rAAV9 particle of any one of the preceding claims.

47. The composition of claim 46, further comprising a pharmaceutically acceptable carrier.

48. A method of delivering a protein of interest to a subject, the method comprising administering to the subject a composition comprising a rAAV9 particle of any one of claims 38-45, wherein the gene of interest encodes a protein of interest.

49. The method of claim 48, wherein the protein of interest is a therapeutic protein or detectable molecule.

50. The method of claim 49, wherein the therapeutic protein is an antibody, a peptibody, a growth factor, a clotting factor, a hormone, a membrane protein, a cytokine, a chemokine, an activating or inhibitory peptide acting on cell surface receptors or ion channels, a cell-permeant peptide targeting intracellular processes, a thrombolytic, an enzyme, a bone morphogenetic protein, a nuclease or other protein used for gene editing, an Fc-fusion protein, or an anticoagulant.

51. The method of claim 49 or 50, wherein the therapeutic protein is a survival motor neuron (SMN) protein.

52. The method of claim 49, wherein the detectable molecule is a fluorescent protein, a bioluminescent protein, or a protein that provides color, or a fragment thereof.

53. The method of any one of claims 48-52, wherein the subject is a mammalian subject.

54. The method of claim 53, wherein the mammalian subject is a human, a non-human primate, non-primate mammal, or mouse.

55. The method of any one of claims 48-54, wherein the subject has or is suspected of having a disease or disorder selected from the group consisting of: cancer, diabetes, autoimmune disease, kidney disease, cardiovascular disease, pancreatic disease, intestinal disease, liver disease, hemophilia, α1-antitrypsin (AAT) deficiency, ischemia, skeletal disease, muscular disease and pulmonary disease.

56. The method of any one of claims 48-55, wherein the subject has or is suspected of having spinal muscular atrophy (SMA).

57. The method of claim 55 or 56, wherein administration of the rAAV9 particle to the subject results in the prevention, alleviation or amelioration of one or more signs or symptoms of the disease or disorder.

58. The method of any one of claims 48-57, wherein the rAAV9 particle is administered to the subject subcutaneously, intraocularly, intravitreally, subretinally, parenterally, intravenously (IV), intracerebro-ventricularly, intramuscularly, intrathecally (IT), intracisternally, orally, intraperitoneally, by oral or nasal inhalation, or by direct injection to one or more cells, tissues, or organs of the subject.

59. A vector comprising a nucleic acid encoding Cap proteins, wherein the Cap proteins form a rAAV9 particle of any one of claims 1-45 when the vector is used to form capsids.

60. A kit comprising the vector of claim 59 and a vector comprising AAV helper genes, wherein the vector of claim 59 and the vector comprising AAV helper genes are provided in a first and second container, wherein the first and second containers are different.

61. The kit of claim 60, further comprising AAV packaging cells that are provided in a third container that is separate from the first and second containers.

62. The kit of claim 60 or 61, wherein the AAV helper genes encode E1, E2, E4 and/or VA helper proteins.

63. A capsid protein of serotype AAV9 comprising a mutation in the D327 amino acid position in SEQ ID NO: 2.

64. The capsid protein of claim 36, wherein the mutation is D327R.

65. A capsid protein of serotype AAV9 comprising a mutation in the D441 amino acid position in SEQ ID NO: 2.

66. The capsid protein of claim 65, wherein the mutation is D441N.

67. A capsid protein of serotype AAV9 comprising a mutation in the T491 amino acid position in SEQ ID NO: 2.

68. The capsid protein of claim 67, wherein the mutation is T491R.

69. A capsid protein of serotype AAV9 comprising a mutation in the V493 amino acid position in SEQ ID NO: 2.

70. The capsid protein of claim 69, wherein the mutation is V493P.

71. A capsid protein of serotype AAV9 comprising a mutation in the R533 amino acid position in SEQ ID NO: 2.

72. The capsid protein of claim 71, wherein the mutation is R533S.

73. A capsid protein of serotype AAV9 comprising a mutation in the N552 amino acid position in SEQ ID NO: 2.

74. The capsid protein of claim 73, wherein the mutation is N552A

75. A capsid protein of serotype AAV9 comprising a mutation in the V553 amino acid position in SEQ ID NO: 2.

76. The capsid protein of claim 75, wherein the mutation is V553A

77. A capsid protein of serotype AAV9 comprising a mutation in the D556 amino acid position in SEQ ID NO: 2.

78. The capsid protein of claim 77, wherein the mutation is D556P.

79. A capsid protein of serotype AAV9 comprising a mutation in the I560 amino acid position in SEQ ID NO: 2.

80. The capsid protein of claim 79, wherein the mutation is I560F.

81. A capsid protein of serotype AAV9 comprising a mutation in the N562 amino acid position in SEQ ID NO: 2.

82. The capsid protein of claim 81, wherein the mutation is N562Y.

83. A capsid protein of serotype AAV9 comprising a mutation in the Q588 amino acid position in SEQ ID NO: 2.

84. The capsid protein of claim 83, wherein the mutation is Q588R.

85. A capsid protein of serotype AAV9 comprising a mutation in the Y706 amino acid position in SEQ ID NO: 2.

86. The capsid protein of claim 85, wherein the mutation is Y706D.

87. A capsid protein of serotype AAV9 comprising a mutation in the N709 amino acid position in SEQ ID NO: 2.

88. The capsid protein of claim 87, wherein the mutation is N709I.

89. The capsid protein of claim 87, wherein the mutation is N709Y.

90. A capsid protein of serotype AAV9 comprising one or more mutations in amino acid positions selected from the group consisting of: D327 and N329 in SEQ ID NO: 2.

91. The capsid protein of claim 90, wherein the one or more mutations are D327T and N329D.

92. A capsid protein of serotype AAV9 comprising one or more mutations in amino acid positions selected from the group consisting of: N552 and V553 in SEQ ID NO: 2.

93. The capsid protein of claim 92, wherein the one or more mutations are N552A and V553A.

94. A capsid protein of serotype AAV9 comprising one or more mutations in amino acid positions selected from the group consisting of: W595 and Q597 in SEQ ID NO: 2.

95. The capsid protein of claim 94, wherein the one or more mutations are W595G and Q597S.

96. A capsid protein of serotype AAV9 comprising one or more mutations in amino acid positions selected from the group consisting of: A656 and P658 in SEQ ID NO: 2.

97. The capsid protein of claim 96, wherein the one or more mutations are: A656G and P658L.

98. A capsid protein of serotype AAV9 comprising one or more mutations in amino acid positions selected from the group consisting of: Y706 and N709 in SEQ ID NO: 2.

99. The capsid protein of claim 98, wherein the one or more mutations are: Y706D and N709I.

100. A capsid protein of serotype AAV9 comprising one or more mutations in amino acid positions selected from the group consisting of: Q588 and T491 in SEQ ID NO: 2.

101. The capsid protein of claim 100, wherein the one or more mutations are: Q588R and T491R.

102. The rAAV9 particle of any one of claims 1-15, wherein the one or more mutations are at amino acid positions T491, D556, N562, Q588, and Y706.

103. The rAAV9 particle of claim 102, wherein the one or more mutations are T491R, D556P, N562Y, Q588R, and Y706D.

104. The rAAV9 particle of claim 102 or 103, further comprising one or more mutations at amino acid positions selected from the group consisting of: T582 and G594.

105. The rAAV9 particle of claim 104, wherein the one or more mutations are amino acid substitution(s) selected from the group consisting of: T582Q and G594E.


SEQUENCE LISTING (8 sequences)

SEQ ID NO: 1 - DNA, 2211 bp, Homo sapiens
  source 1..2211: mol_type = genomic DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 2 - protein, 736 aa, Homo sapiens
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 3 - protein, 10 aa, synthetic construct
NDGSQAVGRS

SEQ ID NO: 4 - protein, 7 aa, synthetic construct
GQNQQTL

SEQ ID NO: 5 - protein, 10 aa, synthetic construct
STTVTQNNNS

SEQ ID NO: 6 - protein, 12 aa, synthetic construct
ASHKEGEDRFFP

SEQ ID NO: 7 - protein, 16 aa, synthetic construct
VDADKVMITNEEEIKT

SEQ ID NO: 8 - protein, 32 aa, synthetic construct
YTSNYYKSNNVEFAVNTEGVYSEPRPIGTRYL